SEQUENCE LISTING (131 sequences)

SEQ ID NO: 1 - DNA, 23 bp, Homo sapiens
agacaaactgggtatcgttgctg

SEQ ID NO: 2 - DNA, 20 bp, Homo sapiens
gtgcctgccaaagcctctac

SEQ ID NO: 3 - DNA, 21 bp, Homo sapiens
  misc_feature (1)..(1): May be modified with a 6-FAM - [6-Carboxyfluorescein] label
ctgatctttatcctccgttcc

SEQ ID NO: 4 - DNA, 24 bp, Homo sapiens
actaccacatgaacatgatgcaca

SEQ ID NO: 5 - DNA, 18 bp, Homo sapiens
ggccacagacagcccaaa

SEQ ID NO: 6 - DNA, 17 bp, Homo sapiens
  misc_feature (1)..(1): May be modified with a 6-FAM - [6-Carboxyfluorescein] label
ctacgtgttcgcagcct

SEQ ID NO: 7 - DNA, 19 bp, Homo sapiens
cgccctcttcttgtggatg

SEQ ID NO: 8 - DNA, 21 bp, Homo sapiens
gaacacggcattcttcctttc

SEQ ID NO: 9 - DNA, 27 bp, Homo sapiens
  misc_feature (1)..(1): May be modified with a 6-FAM - [6-Carboxyfluorescein] label
tctggccaagtttcaactctgctcgct

SEQ ID NO: 10 - DNA, 21 bp, Homo sapiens
tgtggatgttctggccaagtt

SEQ ID NO: 11 - DNA, 22 bp, Homo sapiens
tgaacacggcattcttcctttc

SEQ ID NO: 12 - DNA, 26 bp, Homo sapiens
  misc_feature (1)..(1): May be modified with a 6-FAM - [6-Carboxyfluorescein] label
aactctgctctgctgagaagtccaat

SEQ ID NO: 13 - DNA, 19 bp, Homo sapiens
ggattccccacagctccat

SEQ ID NO: 14 - DNA, 19 bp, Homo sapiens
ctccaagcagacccagcaa

SEQ ID NO: 15 - DNA, 15 bp, Homo sapiens
  misc_feature (1)..(1): May be modified with a 6-FAM - [6-Carboxyfluorescein] label
atgggctacaacttc

SEQ ID NO: 16 - DNA, 17 bp, Homo sapiens
ccggctccgacggtatc

SEQ ID NO: 17 - DNA, 22 bp, Homo sapiens
tgggtctgcttggagagatcat

SEQ ID NO: 18 - DNA, 15 bp, Homo sapiens
  misc_feature (1)..(1): May be modified with a 6-FAM - [6-Carboxyfluorescein] label
accagcagcacaatg

SEQ ID NO: 19 - DNA, 21 bp, Homo sapiens
tgcctgcatttgtacgtgaga

SEQ ID NO: 20 - DNA, 17 bp, Homo sapiens
cctgcgcgaacattgga

SEQ ID NO: 21 - DNA, 16 bp, Homo sapiens
  misc_feature (1)..(1): May be modified with a 6-FAM - [6-Carboxyfluorescein] label
acgctcatgacagcaa

SEQ ID NO: 22 - DNA, 21 bp, Homo sapiens
cctctcactgttgcctgcatt

SEQ ID NO: 23 - DNA, 18 bp, Homo sapiens
agtgcctgcgcgaacatt

SEQ ID NO: 24 - DNA, 31 bp, Homo sapiens
  misc_feature (1)..(1): May be modified with a 6-FAM - [6-Carboxyfluorescein] label
tacgtgagaaacgctcatgacagcaaagtct

SEQ ID NO: 25 - DNA, 25 bp, Homo sapiens
tgcacagagagaaatacccgaatta

SEQ ID NO: 26 - DNA, 19 bp, Homo sapiens
tgcaattcttcggcagcat

SEQ ID NO: 27 - DNA, 26 bp, Homo sapiens
  misc_feature (1)..(1): May be modified with a 6-FAM - [6-Carboxyfluorescein] label
aagtatcgacctcgtcggaaggcgaa

SEQ ID NO: 28 - DNA, 23 bp, Homo sapiens
tcctgcttatccaaattcaccat

SEQ ID NO: 29 - DNA, 28 bp, Homo sapiens
acttccctctgacattacctgataattg

SEQ ID NO: 30 - DNA, 29 bp, Homo sapiens
  misc_feature (1)..(1): May be modified with a 6-FAM - [6-Carboxyfluorescein] label
aagtcgccactggatatcagttcccttgt

SEQ ID NO: 31 - DNA, 26 bp, Homo sapiens
cgtattcatttttttctggaaccttt

SEQ ID NO: 32 - DNA, 22 bp, Homo sapiens
ctgatatccagtggcgacttga

SEQ ID NO: 33 - DNA, 29 bp, Homo sapiens
  misc_feature (1)..(1): May be modified with a 6-FAM - [6-Carboxyfluorescein] label
caggcatttcctgcttatccaaattcacc

SEQ ID NO: 34 - DNA, 22 bp, Homo sapiens
gtgcacctgactcctgaggaga

SEQ ID NO: 35 - DNA, 21 bp, Homo sapiens
ccttgataccaacctgcccag

SEQ ID NO: 36 - DNA, 26 bp, Homo sapiens
  misc_feature (1)..(1): May be modified with a 6-FAM - [6-Carboxyfluorescein] label
aaggtgaacgtggatgaagttggtgg

SEQ ID NO: 37 - DNA, 20 bp, Homo sapiens
ggtaccgggtctccacatga

SEQ ID NO: 38 - DNA, 21 bp, Homo sapiens
gggaaagtcactcacccaagg

SEQ ID NO: 39 - DNA, 25 bp, Homo sapiens
  misc_feature (1)..(1): May be modified with a 6-FAM - [6-Carboxyfluorescein] label
ctgggccagaatcttggtcctcaca

SEQ ID NO: 40 - DNA, 21 bp, Homo sapiens
gcttctctggttggagtcacg

SEQ ID NO: 41 - DNA, 17 bp, Homo sapiens
gcttgctggcggaccct

SEQ ID NO: 42 - DNA, 24 bp, Homo sapiens
  misc_feature (1)..(1): May be modified with a 6-FAM - [6-Carboxyfluorescein] label
ctgcaccaccaaatcatccccgtg

SEQ ID NO: 43 - DNA, 25 bp, Homo sapiens
cttttgctttctgtttcttaagggc

SEQ ID NO: 44 - DNA, 21 bp, Homo sapiens
tcaatctttgggcaggttgaa

SEQ ID NO: 45 - DNA, 24 bp, Homo sapiens
  misc_feature (1)..(1): May be modified with a 6-FAM - [6-Carboxyfluorescein] label
catacgtgcacagggtccccgagt

SEQ ID NO: 46 - DNA, 20 bp, Homo sapiens
ctggtgcccacagttacgct

SEQ ID NO: 47 - DNA, 21 bp, Homo sapiens
aaggatgcgtgactgctatgg

SEQ ID NO: 48 - DNA, 25 bp, Homo sapiens
  misc_feature (1)..(1): May be modified with a 6-FAM - [6-Carboxyfluorescein] label
tcctggcagtgtggtcccttcagaa

SEQ ID NO: 49 - DNA, 24 bp, Homo sapiens
aaagtagacacggccagacttagg

SEQ ID NO: 50 - DNA, 21 bp, Homo sapiens
catccccacatacggaaaaga

SEQ ID NO: 51 - DNA, 27 bp, Homo sapiens
  misc_feature (1)..(1): May be modified with a 6-FAM - [6-Carboxyfluorescein] label
ccctggacactgaaaacaggcaatcct

SEQ ID NO: 52 - DNA, 20 bp, Homo sapiens
cagtcaccccgtgaagtcct

SEQ ID NO: 53 - DNA, 20 bp, Homo sapiens
tttcccccatctgcctattg

SEQ ID NO: 54 - DNA, 27 bp, Homo sapiens
  misc_feature (1)..(1): May be modified with a 6-FAM - [6-Carboxyfluorescein] label
cccatccatcttccctaccagaccagg

SEQ ID NO: 55 - DNA, 24 bp, Homo sapiens
tggtattggctttaaaatactggg

SEQ ID NO: 56 - DNA, 21 bp, Homo sapiens
tgtacccaaaactcagctgca

SEQ ID NO: 57 - DNA, 29 bp, Homo sapiens
  misc_feature (1)..(1): May be modified with a 6-FAM - [6-Carboxyfluorescein] label
tcctcacttctccacccctagttaaacag

SEQ ID NO: 58 - DNA, 25 bp, Homo sapiens
ggtattggctttaaaatactcaacc

SEQ ID NO: 59 - DNA, 23 bp, Homo sapiens
cagctgcaacagttatcaacgtt

SEQ ID NO: 60 - DNA, 20 bp, Homo sapiens
ctggatgcctcactgatcca

SEQ ID NO: 61 - DNA, 22 bp, Homo sapiens
tgggaaggatgcatatgatctg

SEQ ID NO: 62 - DNA, 23 bp, Homo sapiens
  misc_feature (1)..(1): May be modified with a 6-FAM - [6-Carboxyfluorescein] label
ctcccaacccccatttctgcctg

SEQ ID NO: 63 - DNA, 21 bp, Homo sapiens
gctggatgcctcactgatgtt

SEQ ID NO: 64 - DNA, 19 bp, Homo sapiens
gggcacccgtgtgagtttt

SEQ ID NO: 65 - DNA, 22 bp, Homo sapiens
tcagcttgtctgctttctggaa

SEQ ID NO: 66 - DNA, 28 bp, Homo sapiens
  misc_feature (1)..(1): May be modified with a 6-FAM - [6-Carboxyfluorescein] label
tggaggatttctcccctgcttcagacag

SEQ ID NO: 67 - DNA, 21 bp, Homo sapiens
cagcttgtctgctttctgctg

SEQ ID NO: 68 - DNA, 16 bp, Homo sapiens
gccacaagagactcag

SEQ ID NO: 69 - DNA, 20 bp, Homo sapiens
tggcttccttgaggtggaat

SEQ ID NO: 70 - DNA, 28 bp, Homo sapiens
  misc_feature (1)..(1): May be modified with a 6-FAM - [6-Carboxyfluorescein] label
cagtgtcccactcaagtactcctttgga

SEQ ID NO: 71 - DNA, 23 bp, Homo sapiens
ttagagccacaagagacaaccag

SEQ ID NO: 72 - DNA, 20 bp, Homo sapiens
taggattcaaccctggaagc

SEQ ID NO: 73 - DNA, 21 bp, Homo sapiens
ccagcatgcacctgactaaca

SEQ ID NO: 74 - DNA, 28 bp, Homo sapiens
  misc_feature (1)..(1): May be modified with a 6-FAM - [6-Carboxyfluorescein] label
caaggcttcctcaattctccacccttcc

SEQ ID NO: 75 - DNA, 17 bp, Homo sapiens
ccctggatcgccgtgaa

SEQ ID NO: 76 - DNA, 15 bp, Homo sapiens
ccgtgatggcggcca

SEQ ID NO: 77 - DNA, 21 bp, Homo sapiens
cagctgtgtctccggaaactc

SEQ ID NO: 78 - DNA, 17 bp, Homo sapiens
  misc_feature (1)..(1): May be modified with a NED fluorescent label
cttgggcttcctcacct

SEQ ID NO: 79 - DNA, 22 bp, Homo sapiens
acaaggaaacaaaggccaagag

SEQ ID NO: 80 - DNA, 25 bp, Homo sapiens
aattaagcacttcacagagcaggtt

SEQ ID NO: 81 - DNA, 27 bp, Homo sapiens
  misc_feature (1)..(1): May be modified with a 6-FAM - [6-Carboxyfluorescein] label
ttgaaatctgcataccccaggcctcct

SEQ ID NO: 82 - DNA, 27 bp, Homo sapiens
  misc_feature (1)..(1): May be modified with a 6-FAM - [6-Carboxyfluorescein] label
ttgaaatctgcataccccaggcctcct

SEQ ID NO: 83 - DNA, 21 bp, Homo sapiens
aggctgaggcaggagaatctt

SEQ ID NO: 84 - DNA, 17 bp, Homo sapiens
gcagtggcgcgatcttg

SEQ ID NO: 85 - DNA, 24 bp, Homo sapiens
  misc_feature (1)..(1): May be modified with a 6-FAM - [6-Carboxyfluorescein] label
tgaatccaggtggtggaggttgca

SEQ ID NO: 86 - DNA, 29 bp, Homo sapiens
tgagtagtgtttgctaaattcataccttt

SEQ ID NO: 87 - DNA, 18 bp, Homo sapiens
accccaggcctcctgaac

SEQ ID NO: 88 - DNA, 19 bp, Homo sapiens
  misc_feature (1)..(1): May be modified with a 6-FAM - [6-Carboxyfluorescein] label
taagcacttcacagagcag

SEQ ID NO: 89 - DNA, 23 bp, Homo sapiens
gcatggcagacaaactgggtaat

SEQ ID NO: 90 - DNA, 19 bp, Homo sapiens
ctgccaaagcctctaccgg

SEQ ID NO: 91 - DNA, 16 bp, Homo sapiens
  misc_feature (1)..(1): May be modified with a 6-FAM - [6-Carboxyfluorescein] label
ttgctgtctgatcttt

SEQ ID NO: 92 - DNA, 23 bp, Homo sapiens
atgttctggccaagtttcaagat

SEQ ID NO: 93 - DNA, 26 bp, Homo sapiens
gctacagcatagtaggtgttgaagtc

SEQ ID NO: 94 - DNA, 30 bp, Homo sapiens
  misc_feature (1)..(1): May be modified with a 6-FAM - [6-Carboxyfluorescein] label
ctctgctgagaagtccaatcgaaaggaaga

SEQ ID NO: 95 - DNA, 23 bp, Homo sapiens
atgttctggccaagtttcaacgt

SEQ ID NO: 96 - DNA, 26 bp, Homo sapiens
gctacagcatagtaggtgttgaacgc

SEQ ID NO: 97 - DNA, 30 bp, Homo sapiens; identical to SEQ ID NO: 94
  misc_feature (1)..(1): May be modified with a 6-FAM - [6-Carboxyfluorescein] label

SEQ ID NO: 98 - DNA, 24 bp, Homo sapiens
gatgttctggccaagtttcaactc

SEQ ID NO: 99 - DNA, 28 bp, Homo sapiens
ctgctacagcatagtaggtgttgaacac

SEQ ID NO: 100 - DNA, 23 bp, Homo sapiens
atgttctggccaagtttcaacat

SEQ ID NO: 101 - DNA, 26 bp, Homo sapiens
gctacagcatagtaggtgttgaactc

SEQ ID NO: 102 - DNA, 33 bp, Homo sapiens
aataaatcataatgtggatgttctggccaagtt

SEQ ID NO: 103 - DNA, 34 bp, Homo sapiens
aataaatcataatgaacacggcattcttcctttc

SEQ ID NO: 104 - DNA, 20 bp, Homo sapiens
gcgccctcttcttgtggaac

SEQ ID NO: 105 - DNA, 25 bp, Homo sapiens
cattcttcctttcgattggacttct

SEQ ID NO: 106 - DNA, 14 bp, Homo sapiens
  misc_feature (1)..(1): May be modified with a 6-FAM - [6-Carboxyfluorescein] label
tctggccaagtttc

SEQ ID NO: 107 - DNA, 21 bp, Homo sapiens
ttgaacacggcattcttcctt

SEQ ID NO: 108 - DNA, 29 bp, Homo sapiens
  misc_feature (1)..(1): May be modified with a 6-FAM - [6-Carboxyfluorescein] label
caactctgctctgctgagaagtccaatcg

SEQ ID NO: 109 - DNA, 26 bp, Homo sapiens
cacacgctatttctttgcagacttat

SEQ ID NO: 110 - DNA, 25 bp, Homo sapiens
gttgtctagtttcttaccggcaggt

SEQ ID NO: 111 - DNA, 25 bp, Homo sapiens
  misc_feature (1)..(1): May be modified with a 6-FAM - [6-Carboxyfluorescein] label
tgtcacctgatcccttctccgtggc

SEQ ID NO: 112 - DNA, 23 bp, Homo sapiens
acgctatttctttgcagacttgg

SEQ ID NO: 113 - DNA, 25 bp, Homo sapiens
gttgtctagtttcttaccggcagtt

SEQ ID NO: 114 - DNA, 23 bp, Homo sapiens
acgctatttctttgcagactatg

SEQ ID NO: 115 - DNA, 25 bp, Homo sapiens
gttgtctagtttcttaccggcacct

SEQ ID NO: 116 - DNA, 23 bp, Homo sapiens
acgctatttctttgcagactttg

SEQ ID NO: 117 - DNA, 25 bp, Homo sapiens
gttgtctagtttcttaccggcagct

SEQ ID NO: 118 - DNA, 21 bp, Homo sapiens
cagctccatcatgggctacaa

SEQ ID NO: 119 - DNA, 21 bp, Homo sapiens
gcaccagcagcacaatgtaga

SEQ ID NO: 120 - DNA, 22 bp, Homo sapiens
  misc_feature (1)..(1): May be modified with a 6-FAM - [6-Carboxyfluorescein] label
cttgctgggtctgcttggagag

SEQ ID NO: 121 - DNA, 17 bp, Homo sapiens
gcagtgccgcaatctcg

SEQ ID NO: 122 - DNA, 21 bp, Homo sapiens
ctgaggcaggagaattgcttg

SEQ ID NO: 123 - DNA, 15 bp, Homo sapiens
  misc_feature (1)..(1): May be modified with a 6-FAM - [6-Carboxyfluorescein] label
aacctccgcctccca

SEQ ID NO: 124 - DNA, 70 bp, Homo sapiens
gtgcctgccaaagcctctacccgagggaacggaggataaagatcagacagcaacgatacccagtttgtct

SEQ ID NO: 125 - DNA, 32 bp, Homo sapiens
tgttcgcagcctattttgggctgtctgtggcc

SEQ ID NO: 126 - DNA, 41 bp, Homo sapiens
actctgctctgctgagaagtccaatcgaaaggaagaatgcc

SEQ ID NO: 127 - DNA, 33 bp, Homo sapiens
gagcagagttgaaacttggccagaacatccaca

SEQ ID NO: 128 - DNA, 58 bp, Homo sapiens
tctccaagcagacccagcaagctgaagttgtagcccatgatggagctgtggggaatcc

SEQ ID NO: 129 - DNA, 36 bp, Homo sapiens
cattgtgctgctggtgcttgataccgtcggagccgg

SEQ ID NO: 130 - DNA, 32 bp, Homo sapiens
catgacagcaaagtctccaatgttcgcgcagg

SEQ ID NO: 131 - DNA, 40 bp, Homo sapiens
cgctcatgacagcaaagtctccaatgttcgcgcaggcact

(19) United States
(12) Patent Application Publication
(10) Pub. No.: US 2011/0262916 A1
(43) Pub. Date: Oct. 27, 2011

(54) NON-INVASIVE FETAL RHD GENOTYPING FROM MATERNAL WHOLE BLOOD

(76) Inventors: Wen-Hua Fan, San Diego, CA (US); Roger Tim, La Jolla, CA (US); Karena Kosco, Carlsbad, CA (US); Ram Bhatt, San Diego, CA (US)

(21) Appl. No.: 13/054,473

(22) PCT Filed: Jul. 17, 2009
(86) PCT No.: PCT/US09/51061

§ 371 Date: Jul. 8, 2011

Related U.S. Application Data
(60) Provisional application No. 61/082,169, filed on Jul. 18, 2008.

(51) Int. Cl. C12Q 1/68; C07H 21/04

(52) U.S. Cl. 435/6.11; 536/24.33; 536/24.31

(57) ABSTRACT

The present invention discloses methods of determining the RhD genotype of subject. In particular, the invention provides a non-invasive method of determining fetal RhD genotype from a maternal biological sample containing fetal cells. The invention also provides novel probes and primers useful in the described methods. Kits and mixtures comprising the novel probes and primers are also disclosed.

This invention claims priority to U.S. Provisional Application 61/082,169, filed on Jul. 18, 2008, the contents of which are incorporated herein by reference.

### BACKGROUND OF THE INVENTION

Prenatal testing is commonly performed to determine one or more genetic characteristics of the fetus, such as gender, markers of genetic disorders or diseases, and chromosomal abnormalities. One such prenatal test is the determination of fetal Rhesus D antigen (RhD) status. This test is particularly important for pregnant mothers who are RhD-negative. A Rh-D negative mother carrying a RhD-positive fetus can develop antibodies to the RhD antigen expressed on the surface of fetal red blood cells. Because the antibodies are able to pass into the fetal circulation via the placenta, the RhD-positive fetus is at risk for hemolytic disease of the fetus and newborn (HDFN), in which the maternal anti-D antibodies attack the D-positive fetal red blood cells causing them to lyse. The risk of HDFN is significantly increased for subsequent pregnancies in which the fetus is RhD positive. HDFN is characterized by fetal anemia with reticulocytosis in its milder form and fetal lethality in its most severe form.

It is standard antenatal practice to administer a prophylactic anti-RhD immunoglobulin treatment to all RhD-negative pregnant mothers at about 28 weeks gestation with an optional booster at 34 weeks gestation to prevent the development of maternal RhD antibodies to circulating fetal erythrocytes that may express the D surface antigen. However, up to about 38% of these women would be carrying a RhD-negative fetus and be receiving the prophylactic treatment unnecessarily.

Currently available methods to determine fetal RhD status typically entail invasive procedures to obtain fetal cells for testing. For example, chorionic villus sampling (CVS) or aminocentesis can be performed to screen for RhD status or genetic abnormalities. However, spontaneous miscarriage, infection, and alloimmunization are associated with such invasive procedures. Thus, the development of a non-invasive procedure for determining fetal RhD status is desirable to avoid the complications associated with invasive diagnostic tests and the unnecessary administration of expensive prophylactic treatments.

### SUMMARY OF THE INVENTION

The present invention is based, in part, on the development of non-invasive methods for isolating fetal DNA from maternal blood samples and the discovery that detection of specific regions of particular exons of the RhD gene are predictive of RhD genotype. Accordingly, the present invention provides non-invasive methods of determining RhD genotype of a subject, particularly a fetal subject, from a biological sample as well as novel probes and primers for use in the inventive methods.

In one embodiment, the invention provides isolated polynucleotides useful as primers for the amplification of exon 4, exon 5, exon 7, and exon 10 of the human RHD gene. In another embodiment, the invention provides isolated polynucleotides useful as probes for detecting exon 4, exon 5, exon 7, or exon 10 of the RHD gene. The isolated polynucleotides may be dual-labeled probes.

The present invention encompasses methods of determining RHD genotype of a subject. In one embodiment, the method comprises lysing cells in a biological sample to form a lysing mixture, wherein said biological sample contains one or more cells from the subject; extracting nucleic acid from said lysing mixture; and detecting at least one exon of the RHD gene in said extracted nucleic acid, wherein the presence or absence of said exon indicates the subject's RHD genotype. In another embodiment, the subject may be a fetus. The biological sample may be a maternal biological sample containing fetal cells, such as a whole blood sample. In some embodiments, the fetal cells may be preferentially lysed over maternal cells.

In another embodiment, the method comprises detection of at least one exon of the RHD gene by amplifying the at least one exon with one or more primer sets and identifying the at least one exon with one or more labeled probes. The exon may be exon 4, exon 5, exon 7, or exon 10 of the human RHD gene. In another embodiment, two or more primer sets are used to amplify the at least one exon and two or more labeled probes are used to identify the at least one exon. In another embodiment, two or more primer sets amplify a single exon of the human RHD gene. In still another embodiment, two or more primer sets amplify two or more exons of the human RHD gene.

In another embodiment of the invention, the method comprises extracting nucleic acid from a biological sample, wherein the biological sample contains one or more cells from the subject; and detecting at least three exons of the RHD gene in the extracted nucleic acid, wherein the presence or absence of the exons indicates the subject's RHD genotype. In one embodiment, four exons of the RHD gene are detected. The exons that may be detected include exon 4, exon 5, exon 7, and exon 10 of the human RHD gene. The three or more exons of the RHD gene may be detected by amplifying the three or more exons with three or more primer sets and identifying the three or more exons with three or more labeled probes. In some embodiments, the subject is a fetus. In other embodiments, the biological sample is a maternal biological sample containing fetal cells.

In some embodiments, the method further comprises confirming the presence of fetal DNA in said extracted nucleic acid. In one embodiment, the presence of fetal DNA is confirmed by detecting a Y chromosome. In another embodiment, the Y chromosome is detected by amplifying a gene located on the Y chromosome with one or more primer sets, wherein said one or more primer sets comprises a forward primer and a reverse primer; and identifying the gene with one or more labeled probes. In another embodiment, the gene located on the Y chromosome is selected from the group consisting of SRY, FCY, and DAZ. In yet another embodiment, the presence of fetal DNA is confirmed by detecting a paternally-inherited allele.

The present invention also provides a RhD genotyping kit comprising the novel primers and probes disclosed herein. In one embodiment, the kit comprises at least one primer set, wherein said at least one primer set comprises a forward primer and a reverse primer; at least one labeled probe; and instructions for using said at least one primer set and said at least one probe for detecting a RHD gene in a biological sample, wherein said forward primer and said reverse primer hybridize to an exon of the human RHD gene. The exon may be exon 4, exon 5, exon 7, or exon 10 of the human RHD gene. In another embodiment, the kit comprises two or more primer sets and two or more labeled probes. The two or more primer sets may hybridize to a single exon of the human RHD gene or they may hybridize to two or more exons of the human RHD gene. In another embodiment, the kit further comprises a lysis reagent. The lysis reagent may comprise S-(2-Guanidino-4-thiazoyl)-methyl-isothiourea and optionally vitamin E, a detergent, such as triton X-100, Tween-20, NP-40, and saponin.

The present invention also contemplates a reagent mixture comprising isolated nucleic acid and various combinations of the novel probes and primer sets disclosed herein. In one embodiment, the reagent mixture comprises isolated nucleic acid; three or more primer sets for amplification of three or more exons of a RHD gene, wherein each said primer set comprises a forward primer and a reverse primer; and three or more labeled probes. In another embodiment, the reagent mixture comprises isolated nucleic acid; four primer sets for amplification of four exons of a RHD gene; and four labeled probes. The primer sets and labeled probes preferably hybridize to three or more exons selected from exon 4, exon 5, exon 7, and exon 10 of the human RHD gene.

### BRIEF DESCRIPTION OF THE DRAWINGS

FIG. 1. Schematic of Rh antigen genes and corresponding encoded Rh proteins for RhD positive and negative genotypes. Representation of 10 exons of the RhD (red) and RhCE (blue) genes in opposite orientation, the Rh boxes, and the SMP1 gene.

FIG. 2. PCR amplification of exon 4 of RhD gene using primer sets on DNA isolated from blood samples of RhD negative mothers carrying a RhD positive fetus. A. Sample 14202 amplified with RhD primer set 4.2. The top band corresponds to the expected 70 bp amplicon (asterisk). B. Sample 14202 amplified with RhD primer set 4.3. The single band corresponds to the expected 62 bp amplicon (asterisk).

FIG. 3. PCR amplification of exon 5 of RhD gene using primer sets on DNA isolated from blood samples obtained from RhD negative mothers carrying a RhD positive fetus. A. Sample 14180 amplified with RhD primer set 5 (last 2 lanes). The top band corresponds to the expected 83 bp amplicon (asterisk). B. Sample 14180 amplified with RhD primer set 5.2 (last 2 lanes). The top band corresponds to the expected 72 bp amplicon (asterisk).

FIG. 4. PCR amplification of exon 7 of RhD gene using primer sets on DNA isolated from blood samples obtained from RhD negative mothers carrying a RhD positive fetus. A. Sample 14202 amplified with RhD primer set 7 (last 3 lanes). Visible on the 4.5% MS8 agarose gel are two closely sized bands of approximately 53 and 58 bp. Sequencing data confirmed the 58 bp band as the correct amplicon (asterisk). B. Sample 14202 amplified with RhD primer set 7.3 (last 2 lanes). The single band corresponds to the expected 61 bp amplicon (asterisk).

FIG. 5. PCR amplification of exon 10 of RhD gene using primer sets on DNA isolated from blood samples obtained from RhD negative mothers carrying a RhD positive fetus. Sample 14180 was amplified with RhD primer sets for exons 10 (last two lanes) and 10.1 (first two lanes; 10H). The top band corresponds to the correct amplicon for primer set 10 (59 bp, last two lanes) and primer set 10.1 (74 bp, first two lanes).

### DETAILED DESCRIPTION OF THE INVENTION

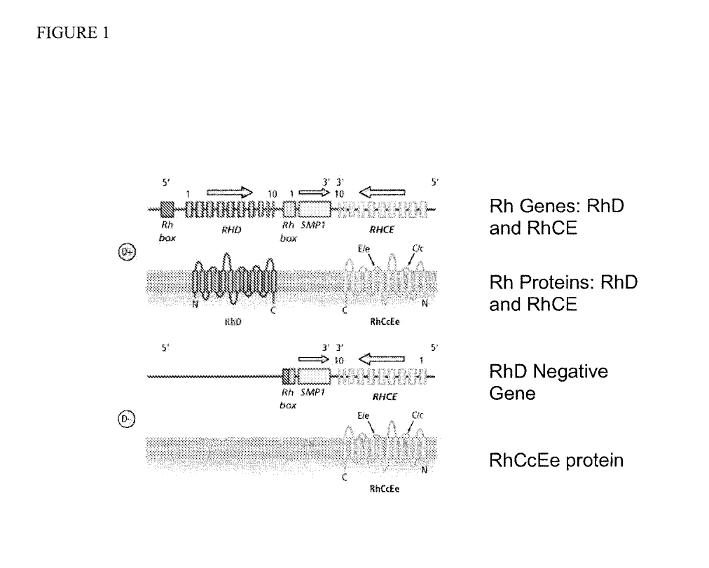

Rhesus (Rh) blood group antigens are considered to be of utmost clinical importance because of their high immunogenicity. Antibodies against Rh antigens are responsible not only for hemolytic disease of the newborn but also for transfusion reactions, and autoimmune hemolytic anemia. Human Rh phenotypes are controlled by two closely linked Rh genes located on chromosome 1 (1p34.1-1p36): RhD, which encodes the D antigen, and RhCE, which encodes the Cc and Ee antigens (Y. Colin et al. (1991) Blood, Vol. 78: 2747). Both genes, which contain 10 exons each with about 94% sequence homology, are in opposite orientation on the chromosome in tail-to-tail configuration, such that the coding strand of the RhD gene is the non-coding strand of RhCE, and vice versa (FIG. 1; N. D. Avent et al. (2006) Expert Reviews in Mol. Med., Vol. 8:1). A small membrane protein (SMP1) gene is located between the two Rh genes. RhD is also flanked by two 9 kb regions of 98.6% homology that are known as Rhesus boxes.

RhD and RhCE encode proteins of 417 amino acids. The RhD and RhCE proteins differ by between 31 and 35 amino acids. RhD encodes for D antigens while RhCE encodes for four common alleles responsible for the expression of the two allelic series of antigens, C/c and E/e. In RhD negative individuals, there is either a complete deletion of the RHD gene or the gene is mutated or partially deleted rendering the gene non-functional such that no RhD antigen is expressed on red blood cells.

About fifteen percent of Caucasians are RhD negative and are usually homozygous for a deletion of RHD. Sixty-six percent of RhD negative Black Africans have an intact RhD, but the gene is inactive due to a non-sense mutation in exon 6 that converts the codon for tyrosine 269 to a translation termination codon. This intact RhD gene, known as RhD pseudogene (RHDΨ), has multiple mutations, including a 37 bp duplication at the intron 3-exon 4 boundary, a missense mutation in exon 5 and a nonsense mutation in exon 6 (B. K. Singleton, et al. (2000) Blood, Vol. 89: 2568). This inactive pseudogene produces no D-protein and no D-antigens. Another non-functional gene that is relatively common among Africans is RhD-CE-D. Despite the presence of RhD exons, no RhD antigens are produced.

As discussed above, awareness that a RhD-negative mother is carrying a RhD-negative fetus would eliminate unnecessary antenatal anti-Rh immunoglobulin prophylaxis and doctors' visits. Standard clinical tests available for determining fetal RhD genotype typically involve the use of invasive procedures that are associated with risks of disrupting the pregnancy. Therefore, there is a need for development of a non-invasive clinical test for determining fetal RhD status.

The present invention is based, in part, on the development of a novel method of isolating fetal DNA from maternal biological samples. As described extensively in co-pending U.S. Provisional Application No. 60/984,698, filed Nov. 1, 2007, which is herein incorporated by reference in its entirety, the method comprises selectively lysing fetal cells over maternal cells by exposing the biological sample to a particular lysing reagent for a specified period of time. Such a method allows for fetal DNA, e.g., high quality fetal DNA to be extracted from the selective lysate. The extracted fetal DNA can be used to screen for various genetic markers, such as RhD genotype.

The present invention is also based on the finding that detection of one or more specific exons of the RHD gene is an accurate predictor of RhD genotype. Accordingly, the present invention provides a method of determining the RHD genotype of a subject. In one embodiment, the method comprises lysing cells in a biological sample to form a lysing mixture, wherein said biological sample contains one or more cells from the subject; extracting nucleic acid from said lysing mixture; and detecting at least one exon of the RHD gene in said extracted nucleic acid, wherein the presence or absence of said exon indicates the subject's RHD genotype. In another embodiment, the subject is a fetus. In another embodiment, said biological sample is a maternal biological sample containing fetal cells. Exemplary maternal biological samples include, but are not limited to, whole blood, plasma, serum, urine, cervical mucus, amniotic fluid, or chorionic villus sample. In a preferred embodiment, said maternal biological sample is a whole blood sample.

Any suitable lysing reagent can be used to lyse cells in a biological sample. Examples of lysing reagents include, but are not limited to, vitamin E, saponin, S-[(2-Guanidino-4-thiazoyl)methyl]-isothiourea (GTMI), or a salt thereof, guanidinium hydrochloride, guanidinium isothiocyanate; urea, lithium ferricyanide, sodium ferricyanide and thiocyanate, potassium ferricyanide and thiocyanate, ammonium chloride, diethylene glycol, Zap-Oglobin and commonly used detergents such as Tritons, Tween, and NP-40, DMSO etc, and any one of the compositions described in U.S. Provisional Application No. 60/984,698, filed Nov. 1, 2007, which is herein incorporated by reference in its entirety.

In one embodiment of the invention, fetal cells are preferentially lysed over maternal cells in a maternal biological sample. The fetal cells may be preferentially lysed over maternal cells by contacting the maternal biological sample with a lysis reagent as described herein for a period of time. Not wishing to be bound by any technical limitation, it is believed that fetal cells in maternal circulation are compromised and are apoptotic in nature, e.g., they can be preferentially lysed at a concentration of lysing reagent that minimally affects the lysis of maternal cells or during a period of time shorter than the time required to lyse maternal cells (if the same concentration of lysing agent is used). Various factors associated with a lysis condition can be varied to preferentially lyse fetal cells. Exemplary factors, including, but not limited to, time period of the lysis reaction, concentration of the lysing agent, nature of the lysing agent, pH of the lysing solution and temperature at which the lysis reaction is carried out can be varied so as to achieve preferential lysing of the fetal cells, but not that of the maternal cells. In another embodiment, said period of time is from about 10 minutes to about 30 minutes. In another embodiment, said lysis reagent comprises S-(2-Guanidino-4-thiazoyl)-methyl-isothiourea (GTMI) or a salt thereof. The concentration of GTMI may be from about 0.1 mM to about 500 mM, more preferably from about 0.5 mM to about 25 mM, and most preferably about 20 mM. In another embodiment, the lysis reagent comprises GTMI, vitamin E, a detergent and optionally saponin. In another embodiment, the lysis reagent comprises GTMI, vitamin E, saponin, triton X-100, DMSO, and a buffer at pH 7.2 to 7.4.

As described above, various factors can be varied to achieve preferential lysis of fetal cells over maternal cells in a maternal biological sample. In one embodiment, the maternal biological sample is contacted with about a 0.1 mM to about a 500 mM GTMI solution for about 1-10 seconds at the high end of the concentration range, and to about an hour at the low end of the concentration range. In another embodiment, the maternal biological sample is contacted with about a 1 mM to about 25 mM GTMI solution for about 5 minutes at the higher end of the concentration range, and to about 30 minutes at the lower end of the concentration range. In yet another embodiment, the biological sample is contacted with about a 1 mM to about 5 mM GTMI solution for about 10-30 minutes. Such variations and manipulations are within the knowledge of one of skill in the art.

Nucleic acid can be extracted from the lysing mixture by any means known in the art. In one embodiment, the nucleic acid is isolated by any suitable means from a supernatant obtained by centrifuging the lysing mixture. The supernatant could, optionally, be further treated before isolating the nucleic acid. For example, the supernatant could be treated with a reagent, e.g., proteinase K that digests proteins and helps clean or purify the nucleic acid in the lysing mixture. Such a reagent, if used, is deactivated, e.g., by heating the sample to about 95° C. The nucleic acid can then be further purified by extractions with, for example chloroform and phenol, and precipitated in ethanol. The nucleic acid pellet can then be suspended in nuclease free water and used for further genetic analysis. Alternatively, the nucleic acid from the supernatant can be cleaned using a commercially available kit, e.g., Roche's Apoptotic DNA Ladder kit, or QIAMP DNA Blood Mini Kit, or Roche's MagNA Pure LC DNA Kit 1.

In one embodiment of the invention, at least one exon of the RHD gene is detected to ascertain the subject's RhD genotype. Any of the ten exons may be detected to determine RhD genotype. Preferably, at least one of exon 4, exon 5, exon 7, or exon 10 is detected. In some embodiments, at least two exons of the RHD gene are detected. In other embodiments, at least three exons of the RHD gene are detected. Detection of all possible combinations of each of the preferred exons are contemplated by the methods of the invention. For example, detection of exons 4 and 5; exons 4 and 7; exons 4 and 10; exons 5 and 7; exons 5 and 10; or exons 7 and 10 may be used to predict a subject's RhD genotype. Similarly, detection of exons 4, 5, and 7; exons 4, 5, and 10; exons 5, 7, and 10, or exons 4, 7, and 10 may be used to diagnose a subject's RhD genotype. In another embodiment, exons 4, 5, 7, and 10 are detected to determine a subject's RhD genotype.

Detection of two or more exons of the RHD gene increases the sensitivity and specificity of the assay. As mentioned previously, variants of the RHD gene are present in the population that contain some or all of the exons of the RHD gene, but due to mutations in the gene do not produce functional D antigen. Individuals carrying such alleles are consequently RhD negative. By detecting two or more exons or specific regions of the exons (such as psi specific regions), false positives due to these non-functional RHDΨ variants can be eliminated. For example, detection of exon 7 would identify both the RHD gene and the non-functional RHDΨ variant. However, detection of the psi specific region of exon 5 would only identify the RHDΨ-gene. In some embodiments, three or more exons of the RHD gene are detected to determine a subject's RhD genotype. In other embodiments, four exons of the RHD gene are detected to determine a subject's RhD genotype.

Exons of the RHD gene may be detected by any method known in the art for identifying the presence of a specific nucleic acid sequence. Suitable methods include, but are not limited to, Southern Blotting, Polymerase Chain Reaction (PCR), Sandwich Hybridization, and Real-Time PCR(RT-PCR). In one embodiment of the invention, detection of at least one exon of the RHD gene comprises amplifying said at least one exon with one or more primer sets and identifying the at least one exon with one or more labeled probes. A “primer set” as used herein refers to a pair of primers, a forward primer and a reverse primer, that flank a specific nucleotide sequence or genomic region and provide free 3′ hydroxyl ends to allow a polymerase to amplify the specific sequence or genomic region. A “labeled probe” refers to a single-stranded nucleic acid conjugated to a compound that produces a detectable signal that is complementary to a target DNA sequence. In another embodiment of the invention, the one or more primer sets amplify exon 4 of the human RHD gene. In another embodiment, the one or more primer sets amplify exon 5 of the human RHD gene. In another embodiment, the one or more primer sets amplify exon 7 of the human RHD gene. In still another embodiment, the one or more primer sets amplify exon 10 of the human RHD gene. Two or more primer sets may be used to amplify specific regions of a single exon. For example, a first primer set may amplify a first region of a first exon, while a second primer set may amplify a second region of the first exon. The first region and second region of an exon may overlap. Alternatively or additionally, two or more primer sets may be used to amplify two different exons. In some embodiments, two or more primer sets amplify two or more exons of the human RHD gene.

As discussed above, a primer set may be designed to amplify a specific exon of the human RED gene or a particular region of that exon. Accordingly, the present invention also provides novel isolated polynucleotides (e.g. oligonucleotides) for use as primers for amplifying particular regions of one or more exons of the human RED gene. The isolated polynucleotide may comprise a sequence selected from the group consisting of SEQ ID NO: 1, SEQ ID NO: 2, SEQ ID NO: 4, SEQ ID NO: 5, SEQ ID NO: 7, SEQ ID NO: 8, SEQ ID NO: 10, SEQ ID NO: 11, SEQ ID NO: 13, SEQ ID NO: 14, SEQ ID NO: 16, SEQ ID NO: 17, SEQ ID NO: 19, SEQ ID NO: 20, SEQ ID NO: 22, and SEQ ID NO: 23, wherein the isolated polynucleotide contains less than fifty bases. In one embodiment, the isolated polynucleotide comprises a sequence selected from the group consisting of SEQ ID NO: 1, SEQ ID NO: 2, SEQ ID NO: 4, SEQ ID NO: 5, SEQ ID NO: 10, SEQ ID NO: 11, SEQ ID NO: 13, SEQ ID NO: 14, SEQ ID NO: 17, SEQ ID NO: 19, SEQ ID NO: 20, SEQ ID NO: 22, and SEQ ID NO: 23, wherein the isolated polynucleotide contains less than fifty bases. The primer polynucleotides may contain one or more chemical modifications including, but not limited to locked nucleic acids (LNA), peptidyl nucleic acids (PNA), sugar modifications, such as 2′-O-alkyl (e.g. 2′-O-methyl, 2′-O-methoxyethyl), 2′-fluoro, and 4′ thio modifications, backbone modifications, such as one or more phosphorothioate, methyl phosphonate, morpholino, or phosphonocarboxylate linkages. In one embodiment, the isolated polynucleotide contains about 10 to about 30 bases. In another embodiment, the isolated polynucleotide contains about 15 to about 25 bases. Preferred primer sets for amplifying exon 4 of the human RHD gene include isolated polynucleotides comprising a sequence recited in SEQ ID NO: 1, SEQ ID NO: 2, SEQ ID NO: 4, or SEQ ID NO: 5. Preferred primer sets for amplifying exon 5 of the human RHD gene include isolated polynucleotides comprising a sequence recited in SEQ ID NO: 7, SEQ ID NO: 8, SEQ ID NO: 10, or SEQ ID NO: 11. Preferred primer sets for amplifying exon 7 of the human RHD gene include isolated polynucleotides comprising a sequence recited in SEQ ID NO: 13, SEQ ID NO: 14, SEQ ID NO: 16, or SEQ ID NO: 17. Preferred primer sets for amplifying exon 10 of the human RHD gene include isolated polynucleotides comprising a sequence recited in SEQ ID NO: 19, SEQ ID NO: 20, SEQ ID NO: 22, or SEQ ID NO: 23. In another embodiment of the invention, the isolated polynucleotide hybridizes to an exon of the human RHD gene. In yet another embodiment, primers of the invention specifically amplify a particular exon or region of an exon of the RHD gene without amplifying any portion of the very closely related RHCE gene or any other gene in the genome, e.g., primers are highly sensitive and able to amplify very small quantities of DNA containing the sequence of the RHD gene in a large background of contaminating chromosomal DNA.

In another embodiment of the invention, the primer polynucleotide may comprise a sequence selected from the group consisting of SEQ ID NO: 76, SEQ ID NO: 77, SEQ ID NO: 79, SEQ ID NO: 80, SEQ ID NO: 83, SEQ ID NO: 84, SEQ ID NO: 86, SEQ ID NO: 87, SEQ ID NO: 89, SEQ ID NO: 90, SEQ ID NO: 92, SEQ ID NO: 93, SEQ ID NO: 95, SEQ ID NO: 96, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 107, SEQ ID NO: 109, SEQ ID NO: 110, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 121, and SEQ ID NO: 122, wherein the primer polynucleotide contains less than fifty bases.

The present invention also provides isolated polynucleotides (e.g. oligonucleotides) for use as probes for detecting one or more exons of the RHD gene. The isolated polynucleotide may comprise a sequence selected from the group consisting of SEQ ID NO: 3, SEQ ID NO: 6, SEQ ID NO: 9, SEQ ID NO: 12, SEQ ID NO: 15, SEQ ID NO: 18, SEQ ID NO: 21, and SEQ ID NO: 24, wherein the isolated polynucleotide contains less than fifty bases. In one embodiment, the isolated polynucleotide contains about 10 to about 40 bases. In another embodiment, the isolated polynucleotide contains about 15 to about 30 bases. Exemplary probe polynucleotides for detecting exon 4 of the RHD gene comprise a sequence recited in SEQ ID NO: 3 or SEQ ID NO: 6. Exemplary probe polynucleotides for detecting exon 5 of the RHD gene comprise a sequence recited in SEQ ID NO: 9 or SEQ ID NO: 12. Exemplary probe polynucleotides for detecting exon 7 of the RHD gene comprise a sequence recited in SEQ ID NO: 15 or SEQ ID NO: 18. Exemplary probe polynucleotides for detecting exon 10 of the RHD gene comprise a sequence recited in SEQ ID NO: 21 or SEQ ID NO: 24.

In another embodiment, the probe polynucleotide may comprise a sequence selected from the group consisting of SEQ ID NO: 78, SEQ ID NO: 81, SEQ ID NO: 82, SEQ ID NO: 85, SEQ ID NO: 88, SEQ ID NO: 91, SEQ ID NO: 94, SEQ ID NO: 97, SEQ ID NO: 106, SEQ ID NO: 108, SEQ ID NO: 111, SEQ ID NO: 120, and SEQ ID NO: 123, wherein the probe polynucleotide contains less than fifty bases.

Preferably the isolated probe polynucleotide contains at least one label that produces a signal that can be detected by one or more methods. Suitable labels include, but are not limited to, radiolabels, such as ³⁵S, ³³P, and ³²P, biotin, digoxigenin, fluorochromes, and enzymes, such as alkaline phosphatase. Additional labels as well as appropriate detection methods for the labels can be ascertained by one of ordinary skill in the art. In one embodiment, the label is attached to the 5′ end of the isolated polynucleotide. In another embodiment, the label is attached to the 3′ end of the isolated polynucleotide. In another embodiment, a first label is attached to the 5′ end of the isolated polynucleotide and a second label is attached to the 3′ end of the isolated polynucleotide. The first label and the second label may interact to generate a unique signal or diminish a signal generated by either label. For example, in the phenomenon known as fluorescence resonance energy transfer or FRET, excitation of a first fluorescent label produces a signal at the emission wavelength of a second fluorescent label located in close proximity to the first fluorescent label due to a transfer of energy. In a variation of this phenomenon, the signal of a first fluorescent label can be quenched by a second label located in close proximity to the first fluorescent label. Use of a first label and a second label that interact in such manners enable the detection of particular conformations of molecules to which the labels are attached. In a preferred embodiment of the invention, the isolated probe polynucleotide contains a reporter molecule attached to the 5′ end of the polynucleotide and a quencher molecule attached to the 3′ end of the polynucleotide. Any reporter/quencher combination may be conjugated to the isolated polynucleotide. Suitable reporter molecules include, but are not limited to, 6-carboxyfluorescein (6-FAM), tetrachlorofluorescein (TET), ROX, HEX, and JOE. Suitable quencher molecules include, but are not limited to tetramethylrhodamine (TAMRA), dihydrocyclopyrroloindole tripeptide minor groove binder (MGB), black hole quencher (BHQ), and minor groove binding nonfluorescent quencher (MGBNFQ). Such dual-labeled polynucleotides are particularly useful as probes in combination with the inventive primer polynucleotides for detection of one or more exons of the RHD gene using real-time PCR techniques.

The present invention encompasses methods of determining RHD genotype of a fetus comprising lysing cells in a maternal biological sample containing fetal cells to form a lysing mixture; extracting nucleic acid from said lysing mixture; and detecting at least one exon of the RHD gene in said extracted nucleic acid, wherein the presence or absence of said exon indicates the fetus' RhD genotype. In one embodiment, the method further comprises confirming the presence of fetal DNA in said extracted nucleic acid.

In some embodiments, the presence of fetal DNA in the extracted nucleic acid is confirmed by detecting a Y chromosome. Several methods are known to those skilled in the art for detecting a Y chromosome in DNA extracted from biological samples. In one embodiment, the Y chromosome is detected by amplifying a gene located on the Y chromosome with one or more primer sets, wherein said one or more primer sets comprises a forward primer and a reverse primer; and identifying the gene with one or more labeled probes. Any one of the genes located on the human Y chromosome may be detected including AMELY (amelogenin, Y-chromosomal), ANT3Y (adenine nucleotide translocator-3 on the Y), ASMTY (which stands for acetylserotonin methyltransferase), AZF1 (azoospermia factor 1), AZF2 (azoospermia factor 2), BPY2 (basic protein on the Y chromosome), CSF2RY (granulocyte-macrophage colony-stimulating factor receptor, alpha subunit on the Y chromosome), DAZ (deleted in azoospermia), IL3RAY (interleukin-3 receptor), PRKY (protein kinase, Y-linked), RBM1 (RNA binding motif protein, Y chromosome, family 1, member A1), RBM2 (RNA binding motif protein 2), RPS4Y (Ribosomal protein S4, Y-linked copy 1), RPS4Y2 (Ribosomal protein S4, Y-linked copy 2), SRY (sex-determining region), TSPY (testis-specific protein), UTY (ubiquitously transcribed TPR gene on Y chromosome), ZFY (zinc finger protein), and FCY. In a preferred embodiment, the gene is SRY, FCY, or DAZ. Exemplary primers for amplifying the SRY gene include polynucleotides comprising the sequence recited in SEQ ID NO: 25 or SEQ ID NO: 26. Exemplary primers for amplifying the FCY gene include polynucleotides comprising the sequence recited in SEQ ID NO: 28 or SEQ ID NO: 29. Exemplary primers for amplifying the DAZ gene include polynucleotides comprising the sequence recited in SEQ ID NO: 31 or SEQ ID NO: 32. Exemplary probes for detecting the SRY gene include polynucleotides comprising SEQ ID NO: 27. Exemplary probes for detecting the FCY gene include polynucleotides comprising SEQ ID NO: 30. Exemplary probes for detecting the DAZ gene include polynucleotides comprising SEQ ID NO: 33.

The present invention also provides novel polynucleotides (e.g. oligonucleotides) for use as exemplary primers and probes for detection of the DAZ gene on the Y chromosome. In one embodiment, the isolated polynucleotide comprises a sequence recited in SEQ ID NO: 31, SEQ ID NO: 32, or SEQ ID NO: 33, wherein the isolated polynucleotide contains less than fifty bases. In another embodiment, the isolated polynucleotide contains about 10 to about 30 bases. In another embodiment, the isolated polynucleotide contains about 15 to about 25 bases.

In other embodiments, the presence of fetal DNA in the extracted nucleic acid is confirmed by detecting a paternally-inherited allele. Detection of a paternally-inherited allele may be performed by determining the presence of one or more polymorphic markers. In one embodiment, DNA obtained from maternal cells is screened for one or more polymorphic markers. Subsequently, the extracted DNA obtained from selective lysis of the maternal blood samples is screened for one or more polymorphic markers not found in the maternal DNA extracts. The presence of one or more polymorphic markers in the extracts from the selected lysis is indicative of a paternally-inherited allele and confirms the presence of fetal DNA in the extract. Various polymorphic markers can be used to determine paternally-inherited alleles. Primers and probes can be designed to detect appropriate polymorphic markers in extracted DNA. An exemplary set of polymorphic markers and primers and probes for their detection is described in Example 3.

The present invention also encompasses methods of determining RHD genotype of a subject by detecting multiple exons of the RHD gene in extracted nucleic acid from a biological sample obtained from the subject. In one embodiment, the method comprises extracting nucleic acid from a biological sample, wherein said biological sample contains one or more cells from the subject; and detecting at least three exons of the RHD gene in said extracted nucleic acid, wherein the presence or absence of said exons indicates the subject's RhD genotype. In another embodiment, four exons of the RHD gene are detected in said extracted nucleic acid. In some embodiments, the subject is a fetus. In other embodiments, the biological sample is a maternal biological sample containing fetal cells.

Detection of any combination of the ten exons of the RHD gene may be used to ascertain a subject's RhD genotype. Preferably, a combination of exon 4, exon 5, exon 7, and exon 10 are detected. In some embodiments, all four of exon 4, exon 5, exon 7, and exon 10 are detected to determine a subject's RhD genotype. Detection of the three or more exons of the RHD gene may comprise amplification of the three or more exons with three or more primer sets and identification of the three or more exons with three or more labeled probes or any other method described above. The three or more primer sets and three or more labeled probes may be any of the inventive primers and probes described herein.

The present invention also provides a RhD genotyping kit comprising the novel primer sets and novel probes described herein. In one embodiment, the kit comprises at least one primer set, wherein said at least one primer set comprises a forward primer and a reverse primer; at least one labeled probe; and instructions for using said at least one primer set and said at least one probe for detecting a RHD gene in a biological sample, wherein said forward primer and said reverse primer hybridize to an exon of the human RHD gene.

In some embodiments, the kit comprises two or more primer sets and two or more labeled probes. In other embodiments, the two or more primer sets hybridize to a single exon of the human RHD gene. In still other embodiments, the two or more primer sets hybridize to two or more exons of the human RHD gene. Preferably each primer set comprises a forward primer and a reverse primer for amplifying an exon of the RHD gene. In one embodiment, the exon is exon 4, exon 5, exon 7, or exon 10 of the human RHD gene. In another embodiment, the forward primers may include a polynucleotide comprising the sequence recited in SEQ ID NO: 1, SEQ ID NO: 4, SEQ ID NO: 7, SEQ ID NO: 10, SEQ ID NO: 13, SEQ ID NO: 16, SEQ ID NO: 19 or SEQ ID NO: 22. In another embodiment, the reverse primers may include a polynucleotide comprising the sequence recited in SEQ ID NO: 2, SEQ ID NO: 5, SEQ ID NO: 8, SEQ ID NO: 11, SEQ ID NO: 14, SEQ ID NO: 17, SEQ ID NO: 20 or SEQ ID NO: 23. In yet another embodiment, the labeled probes may include a polynucleotide comprising the sequence recited in SEQ ID NO: 3, SEQ ID NO: 6, SEQ ID NO: 9, SEQ ID NO: 12, SEQ ID NO: 15, SEQ ID NO: 18, SEQ ID NO: 21 or SEQ ID NO: 24.

In another embodiment, the kit further comprises a lysing reagent. The lysing reagent may be any of the lying reagents for lysing biological cells including those described herein. In a preferred embodiment, the lysis reagent comprises S-(2-Guanidino-4-thiazoyl)-methyl-isothiourea. In another embodiment, the lysis reagent comprises S-(2-Guanidino-4-thiazoyl)-methyl-isothiourea, vitamin E, triton X-100, and saponin. In yet another embodiment, the lysis reagent comprises S-(2-Guanidino-4-thiazoyl)-methyl-isothiourea, vitamin E, saponin, DMSO, triton X-100 and a buffer at pH 7.2 to 7.4. The kit may further comprise instructions for using the lysis reagent to lyse cells in a biological sample and subsequently prepare DNA extracts from the lysate. In another embodiment, the instructions may describe the use of the lysis reagent for selectively lysing fetal cells in a maternal biological sample.

The present invention also contemplates a reagent mixture comprising isolated nucleic acid and various combinations of the novel primers and probes described herein. In one embodiment, the reagent mixture comprises isolated nucleic acid; three or more primer sets for amplification of three or more exons of a RHD gene, wherein each said primer set comprises a forward primer and a reverse primer; and three or more labeled probes. “Isolated nucleic acid” refers to nucleic acid extracted from a biological sample of a subject. The isolated nucleic acid can serve as a template for the amplification of specific exons of the RHD gene by the inventive primers included in the reagent mixture. In a preferred embodiment, the three or more exons are selected from the group consisting of exon 4, exon 5, exon 7, and exon 10 of the human RHD gene. In another embodiment, the reagent mixture comprises isolated nucleic acid; four primer sets for amplification of four exons of a RHD gene; and four labeled probes. The four exons may be exon 4, exon 5, exon 7, and exon 10 of the human RHD gene.

As described in detail above, the novel primers and probes of the invention specifically amplify particular exons or regions of particular exons of the RHD gene. Any of the aforementioned primer polynucleotides and probe polynucleotides may be included in the reagent mixture. In some embodiments, the forward primers of the three or more primer sets are selected from the group consisting of SEQ ID NO: 1, SEQ ID NO: 4, SEQ ID NO: 7, SEQ ID NO: 10, SEQ ID NO: 13, SEQ ID NO: 16, SEQ ID NO: 19, and SEQ ID NO: 22. In other embodiments, the reverse primers of the three or more primer sets are selected from the group consisting of SEQ ID NO: 2, SEQ ID NO: 5, SEQ ID NO: 8, SEQ ID NO: 11, SEQ ID NO: 14, SEQ ID NO: 17, SEQ ID NO: 20, and SEQ ID NO: 23. In still other embodiments, the three or more labeled probes are selected from the group consisting of SEQ ID NO: 3, SEQ ID NO: 6, SEQ ID NO: 9, SEQ ID NO: 12, SEQ ID NO: 15, SEQ ID NO: 18, SEQ ID NO: 21, and SEQ ID NO: 24.

This invention is further illustrated by the following additional examples that should not be construed as limiting. The contents of all references, patents, and published patent applications cited throughout this application, as well as the Figures, are incorporated herein by reference in their entirety.

### EXAMPLES

### Example 1

### RhD Genotyping of Fetal Cells Obtained from Maternal Blood Lysates

Blood samples (20 ml) from pregnant women (8 to 12 weeks gestation) were treated with lysis reagent (2 ml) for 10-20 minutes at room temperature with gentle mixing in a 50 ml capped tube. The lysis reagent contained 10 mM S-(2-Guanidino-4-thiazoyl)-methyl-isothiourea (GTMI), 5 mM Vitamin E, 1% Triton X-100, 0.5% Saponin, 2.5% DMSO, 0.15 M NaCl, and 0.05 M HEPES, pH 7.2. The tube was then centrifuged at 2000 g for 10 minutes and the supernatant transferred into another 50 ml tube. This procedure results in preferential lysis of apoptotic fetal cells over maternal cells present in the maternal blood sample. See co-pending U.S. Provisional Application No. 60/984,698, filed Nov. 1, 2007, which is herein incorporated by reference in its entirety.

To dissociate any proteins from DNA, the supernatant was first treated with proteinase K (10 mg/ml) at 55° C. for 10 minutes, followed by treatment with lysis buffer (either 12 ml of 5 mM guanidinium isothiocyanate, 20% triton X-100 in 50 mM Tris.HCl (pH 7.2) or 12 ml, MagNA Pure Kit, Roche Diagnostics) and Magnetic Glass Particles (MGPs) (3 ml, MagNA Pure Kit). After a thorough mixing the tube was rotated on a rotating wheel at room temperature for 20-30 minutes. The supernatant tube was placed on a magnetic rack for 2 minutes to congregate DNA bound to MGPs. After MGP congregation, the supernatant was discarded and the MGPs were washed twice with Wash Buffer I and twice with Wash Buffer II or until the washings became clear. The MGPs were completely air-dried for several hours. DNA was eluted from the beads with the elution buffer (2×400 μL) and the concentration determined on NanoDrop Spectrophotometer-1000. The eluted DNA was employed for PCR amplification to determine fetal origin and fetal RhD status as described below.

To improve specificity, two sets of primers and probes for each of four exons (4, 5 7, and 10) of the RhD gene were developed. Confirmation of the fetal RhD status from the DNA extracted from maternal blood was made by real time-polymerase chain reaction (RT-PCR) on the matching fetal tissue DNA. DNA from known RhD positive and RhD negative individuals was used as a control. The sequences of primers and probes that were developed for each exon of the human RHD gene for use in quantitative PCR methods to assess fetal genotypes are listed below:

5′-----------------------------------------------3′
RhD Exon 4.2 Forward Primer
(SEQ ID NO: 1)
AGACAAACTGGGTATCGTTGCTG
RhD Exon 4.2 Reverse Primer
(SEQ ID NO: 2)
GTGCCTGCCAAAGCCTCTAC
RhD Exon 4.2 Probe
(SEQ ID NO: 3)
(6FAM)-CTGATCTTTATCCTCCGTTCC-(BHQ)
RhD Exon 4.3 Forward Primer
(SEQ ID NO: 4)
ACTACCACATGAACATGATGCACA
RhD Exon 4.3 Reverse Primer
(SEQ ID NO: 5)
GGCCACAGACAGCCCAAA
RhD Exon 4.3 Probe
(SEQ ID NO: 6)
(6FAM)-CTACGTGTTCGCAGCCT-(BHQ)
RhD Exon 5 Forward Primer
(SEQ ID NO: 7)
CGCCCTCTTCTTGTGGATG
RhD Exon 5 Reverse Primer
(SEQ ID NO: 8)
GAACACGGCATTCTTCCTTTC
RhD exon 5 Probe
(SEQ ID NO: 9)
(6FAM)-TCTGGCCAAGTTTCAACTCTGCTCGCT-(BHQ)
RhD Exon 5.2 Forward Primer
(SEQ ID NO: 10)
TGTGGATGTTCTGGCCAAGTT
RhD Exon 5.2 Reverse Primer
(SEQ ID NO: 11)
TGAACACGGCATTCTTCCTTTC
RhD Exon 5.2 Probe
(SEQ ID NO: 12)
(6FAM)-AACTCTGCTCTGCTGAGAAGTCCAAT-(BHQ)
RhD Exon 7 Forward Probe
(SEQ ID NO: 13)
GGATTCCCCACAGCTCCAT
RhD Exon 7 Reverse Probe
(SEQ ID NO: 14)
CTCCAAGCAGACCCAGCAA
RhD Exon 7 Probe
(SEQ ID NO: 15)
(6FAM)-ATGGGCTACAACTTC-(MGBNFQ)
RhD Exon 7.3 Forward Probe
(SEQ ID NO: 16)
CCGGCTCCGACGGTATC
RhD Exon 7.3 Reverse Probe
(SEQ ID NO: 17)
TGGGTCTGCTTGGAGAGATCAT
RhD Exon 7.3 Probe
(SEQ ID NO: 18)
(6FAM)-ACCAGCAGCACAATG-(BHQ)
RhD Exon 10 Forward Probe
(SEQ ID NO: 19)
TGCCTGCATTTGTACGTGAGA
RhD Exon 10 Reverse Probe
(SEQ ID NO: 20)
CCTGCGCGAACATTGGA
RhD Exon 10 Probe
(SEQ ID NO: 21)
(6FAM)-ACGCTCATGACAGCAA-(BHQ)
RhD Exon 10.1 Forward Primer
(SEQ ID NO: 22)
CCTCTCACTGTTGCCTGCATT
RhD Exon 10.1 Reverse Primer
(SEQ ID NO: 23)
AGTGCCTGCGCGAACATT
RhD Exon 10.1 Probe
(SEQ ID NO: 24)
(6FAM)-TACGTGAGAAACGCTCATGACAGCAAAGTCT-(BHQ)

All RT-PCR reactions were performed in triplicate. At least two of the three reactions were required to result in PCR amplification products before determining fetal RhD genotypes. The composition of the PCR reaction mixture and the cycling protocol are shown below. All RT-PCR reactions were performed and analyzed using ABI's 7900HT Fast Real-Time PCR System.

### Composition of the PCR Reaction Mixture:

DNA=7.5 ul
Taqman Universal PCR Master Mix=12.5 ul
RhD exon Forward Primer=1.25 ul (0.3 pmol/ul)
RhD exon Reverse Primer=1.25 ul (0.3 pmol/ul)
RhD Exon Probe=2.5 ul (0.15 pmol/ul)
Total PCR Reaction Volume=25 ul

### PCR Cycle:

Polymerase activation at 95° C. for 10 minutes
Annealing at 60° C. for 60 seconds
Extension at 60° C. for 10 seconds
Denaturation at 95° C. for 15 seconds
45 Cycles

RT-PCR results were tabulated by cycle threshold (Ct) values for each PCR reaction. The higher the amount of initial DNA template, the lower the Ct value. Low Ct values (<30) were indicative of maternal RhD positive status (in 1-3% of samples a positive signal was obtained from maternal DNA due to the presence of non-functional genetic variants of the RhD gene). High Ct values (34 to 43) were diagnostic of positive fetal RhD status, and also confirmed the presence of fetal DNA in the lysate. No amplification of template (extracted) DNA was interpreted as the fetus lacking functional D antigen (i.e. fetal RhD negative), which was confirmed by the RT-PCR of DNA extracted from the matching fetal tissues. Table 1 lists the RT-PCR Ct values of DNA extracted from 16 RhD negative maternal blood samples. Table 2 summarizes the results.

TABLE 1
Cycle threshold values for RHD exons amplified from DNA extracted from RhD-
negative maternal blood samples

| Sample | | Fetal | Ct Values RT-PCR | | | | | | | | | | | |
|---|---|---|---|---|---|---|---|---|---|---|---|---|---|---|
| ID | GA | Tissu | Exon 4.2 | | | Exon 4.3 | | | Exon 5 | | | Exon 5.2 | | |
| 13560 | 9.0 | neg | neg | neg | neg | neg | neg | neg | neg | neg | neg | neg | neg | neg |
| 13577 | 9.0 | pos | 35.5 | 34.5 | 35.2 | 35.1 | 34.7 | 34.8 | 35.5 | 36.1 | 35.8 | 34.0 | 34.5 | 34.0 |
| 13580 | 11.0 | pos | neg | neg | neg | 44.0 | 43.7 | 43.6 | neg | neg | neg | neg | neg | neg |
| 13613 | 7.0 | pos | neg | neg | neg | neg | neg | neg | neg | neg | neg | neg | neg | neg |
| 13634 | 8.0 | pos | 40.4 | 41.1 | 41.4 | 37.2 | 37.0 | 37.6 | 38.7 | 37.5 | 38.0 | neg | neg | neg |
| 13661 | 9.0 | neg | neg | neg | neg | neg | neg | neg | neg | neg | neg | neg | neg | neg |
| 13667 | 18.0 | pos | 36.5 | 36.7 | 37.0 | 35.7 | 34.9 | 35.5 | 36.9 | 35.1 | 36.2 | 34.5 | 34.6 | 35.0 |
| 13679 | 9.0 | neg | neg | neg | neg | neg | neg | neg | neg | neg | neg | neg | neg | neg |
| 13690 | 6.0 | pos | neg | neg | neg | 29.8 | 29.5 | 29.2 | 42.5 | neg | 44.7 | 41.7 | neg | neg |
| 13699 | 7.0 | pos | 42.6 | neg | neg | 41.1 | 40.1 | 40.6 | neg | 44.5 | neg | 37.2 | neg | neg |
| 13701 | 9.0 | pos | 35.2 | 36.3 | 35.1 | 34.2 | 35.4 | 35.0 | 35.3 | 34.6 | 33.7 | 31.6 | 37.8 | 37.8 |
| 13709 | 9.0 | pos | 36.0 | 36.5 | 36.4 | 36.6 | 38.3 | 35.8 | 39.0 | 37.5 | 38.4 | 34.5 | 34.8 | 35.1 |
| 13711 | 12.0 | pos | 36.3 | 36.3 | 35.9 | 35.9 | 35.9 | 37.0 | 43.1 | 39.8 | 40.3 | 33.5 | 33.0 | 35.0 |
| 13713 | 12.0 | pos | 28.7 | 28.5 | 28.6 | 28.4 | 28.6 | 28.7 | 28.5 | 28.6 | 28.5 | 29.0 | 28.5 | 28.0 |
| 13722 | 11.0 | pos | 34.3 | 36.3 | 35.8 | 35.8 | 36.7 | 25.8 | 36.0 | 35.2 | 33.8 | 34.0 | 35.0 | 34.3 |
| 13743 | 9.0 | pos | neg | 38.0 | 38.2 | neg | neg | neg | 39.0 | 39.3 | neg | neg | neg | neg |
| Sample | | Fetal | Ct Values RT-PCR | | | | | | | | | | | |
| ID | GA | Tissu | Exon 7 | | | Exon 7.3 | | | Exon 10 | | | Exon 10.1 | | |
| 13560 | 9.0 | neg | neg | neg | neg | neg | neg | neg | neg | neg | neg | neg | neg | neg |
| 13577 | 9.0 | pos | 34.2 | 35.5 | 35.0 | 34.1 | 35.2 | 35.0 | 33.2 | 33.9 | 34.0 | 36.7 | 37.5 | 37.0 |
| 13580 | 11.0 | pos | 36.2 | 36.6 | 36.0 | neg | neg | neg | neg | neg | neg | neg | neg | neg |
| 13613 | 7.0 | pos | 42.2 | 41.6 | 41.0 | 41.7 | 42.4 | 41.0 | neg | neg | neg | neg | neg | neg |
| 13634 | 8.0 | pos | 35.2 | 35.6 | 36.0 | 37.8 | 38.3 | 38.0 | 36.6 | 37.5 | 37.0 | neg | neg | neg |
| 13661 | 9.0 | neg | neg | neg | neg | neg | neg | neg | neg | neg | neg | neg | neg | neg |
| 13667 | 18.0 | pos | 33.6 | 33.1 | 34.0 | 37.1 | 36.7 | 37.0 | 35.9 | 34.9 | 35.0 | 36.7 | 36.7 | 37.0 |
| 13679 | 9.0 | neg | neg | neg | neg | neg | neg | neg | neg | neg | neg | neg | neg | neg |
| 13690 | 6.0 | pos | 25.5 | 26.8 | 25.9 | 29.9 | 30.0 | 30.2 | 31.1 | 32.8 | 29.9 | 33.2 | 32.2 | 31.6 |
| 13699 | 7.0 | pos | 35.7 | 36.5 | 39.5 | neg | 42.3 | 42.0 | neg | 41.3 | 41.8 | 43.5 | neg | neg |
| 13701 | 9.0 | pos | 31.9 | 31.7 | 31.5 | 35.4 | 35.2 | 36.0 | 34.0 | 33.1 | 33.6 | 34.6 | 35.1 | 35.7 |
| 13709 | 9.0 | pos | 36.1 | 35.8 | 35.5 | 37.4 | 36.6 | 37.3 | 32.3 | 35.9 | 35.7 | 38.4 | neg | neg |
| 13711 | 12.0 | pos | 32.2 | 31.1 | 32.0 | 37.5 | 38.1 | 38.4 | 37.6 | 36.1 | 35.7 | 38.8 | 37.3 | 37.5 |
| 13713 | 12.0 | pos | 26.6 | 26.5 | 26.5 | 29.0 | 29.0 | 29.7 | 27.2 | 27.1 | 26.9 | 28.9 | 29.0 | 28.9 |
| 13722 | 11.0 | pos | 32.6 | 32.6 | 33.4 | 36.2 | 36.0 | 36.3 | 34.9 | 34.1 | 34.8 | 35.6 | 36.6 | 35.7 |
| 13743 | 9.0 | pos | neg | neg | neg | neg | neg | neg | neg | neg | neg | neg | neg | neg |

TABLE 2
Summary of Results. Fetal RhD status was
correctly identified in all 16 cases.

Concordant Fetal Tissue

| | RhD-Positive | RhD-Negative |
|---|---|---|
| RhD-Positive | 13 | 0 |
| RhD-Negative | 0 | 3 |

* Sensitivity: 100% (no false negative)
* Specificity: 100% (no false positive)

### Example 2

### Validation of RhD Exon Primer Sets

Occasionally, high Ct values of greater than 37 were found when RT-PCR amplification was performed on selectively lysed blood from an RhD negative mother. To verify that these high Ct values of amplicons generated by RhD primers were in fact representative of PCR products corresponding to the presence of small amounts of RhD positive fetal DNA, the PCR product size was examined by gel electrophoresis and subsequently sequenced to verify that the correct RhD locus had been amplified.

Blood samples from RhD negative mothers carrying an RhD positive fetus (samples 14180 or 14202) were lysed according to the method described in Example 1. Following lysis, DNA was isolated using the Roche MagNA Pure kit. Real-time PCR was performed on ABI's 7900HT RT-PCR System using the Taqman Universal Master Mix (Applied Biosystems) with RhD primer sets 4.2, 4.3, 5, 5.2, 7, 7.3, 10, and 10.1 described in Example 1. For every primer set, a no DNA template control (NTC) was run, which always resulted in an undetermined Ct value indicating no amplification. High Ct (>37) PCR reactions and NTCs were then run on either a 1.5% agarose gel or a 4.5% agarose MS-8 gel against a known DNA ladder as a marker to assess the size of amplicons. Expected amplicon size, 58 to 83 bp long, was observed for all PCR products resulting from all the RhD primers.

After extraction of the expected amplicon bands from the gel, the amplicons were sequenced directly using either the forward or reverse PCR primers. Sequencing was performed by Retrogen in San Diego. If the sequencing data obtained was inconclusive, the amplicon was cloned into the pCR4-TOPO vector (Invitrogen) and sequenced using the T3 primer. Six of the eight amplicons could be sequenced directly, while the remaining two had to be sequenced from the pCR4-TOPO vector. The sequence obtained for each of the amplicons was compared to the sequence published in the GenBank sequence database (NCBI) using the BLAST algorithm. Each primer set is discussed below. DNA sequence in BLACK LETTERS represents the amplicon sequence obtained experimentally. DNA sequence in RED LETTERS is the sequence published in GenBank (indicated below as Gene Bank). In all cases, the sequence of the PCR amplicon was a 100% match to the RhD locus.

### Rh(D) Primer Set 4.2 (Exon 4)

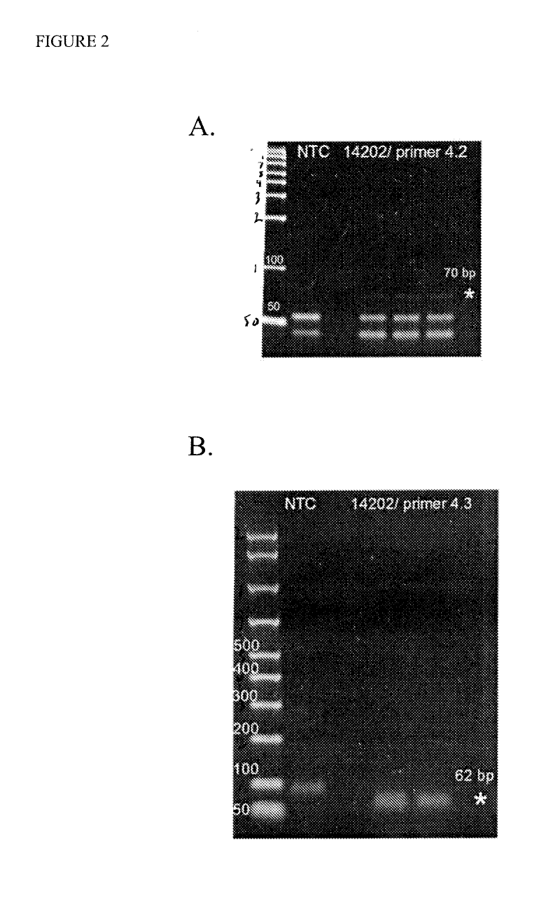

FIG. 2A shows sample 14202 amplified with primer set 4.2 (Ct 40.2). The three PCR products observed were cloned separately into pCR4-TOPO and sequenced. The top band corresponds to the correct 70 bp amplicon, which was identified using BLAST as the RhD locus:

Sequencing: Homo sapiens Rh Blood Group, D Antigen (RHD), mRNA

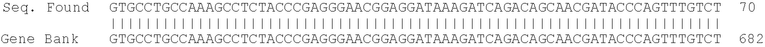

### Rh(D) Primer Set 4.3 (Exon 4)

FIG. 2B depicts the electrophoretic gel of sample 14202 amplified with RhD primer set 4.3 (Ct 40.4).

Sequencing: Homo sapiens Rh Blood Group, D Antigen (RHD), mRNA

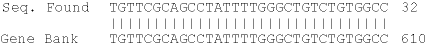

### Rh(D) Primer Set 5 (Exon 5)

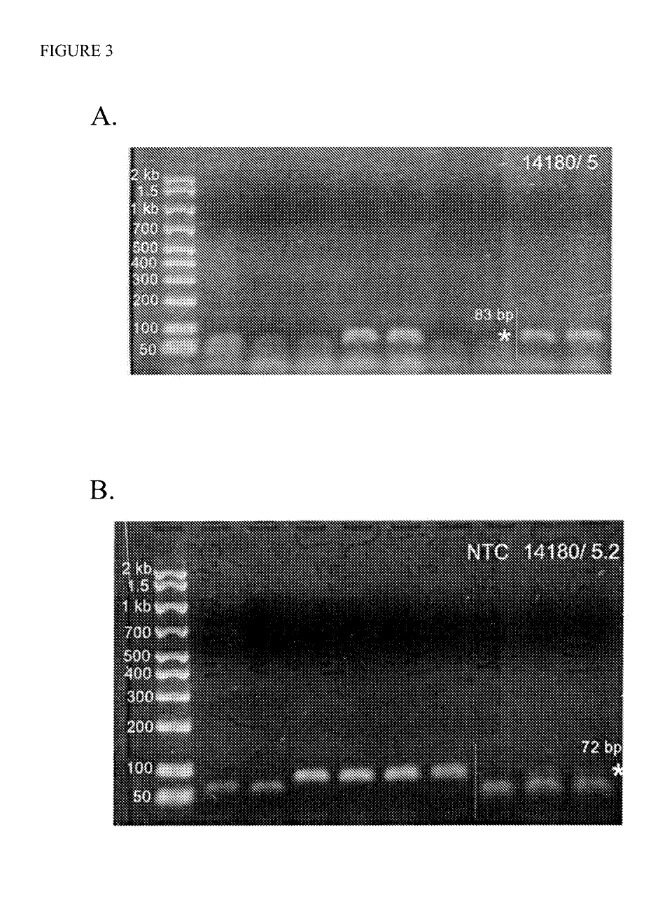

FIG. 3A shows the electrophoretic gel of sample 14180 amplified with RhD primer set 5 (last 2 lanes) (Ct 39.3).

Sequencing: Homo sapiens Rh Blood Group, D Antigen (RHD), mRNA

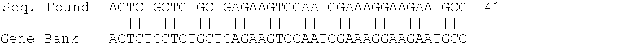

### Rh(D) Primer Set 5.2 (Exon 5)

FIG. 3B shows the amplification of sample 14180 with RhD primer set 5.2 (last 2 lanes) (Ct 35).

Sequencing: Homo sapiens Rh Blood Group, D Antigen (RHD), mRNA

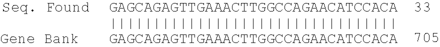

### Rh(D) Primer Set 7 (Exon 7)

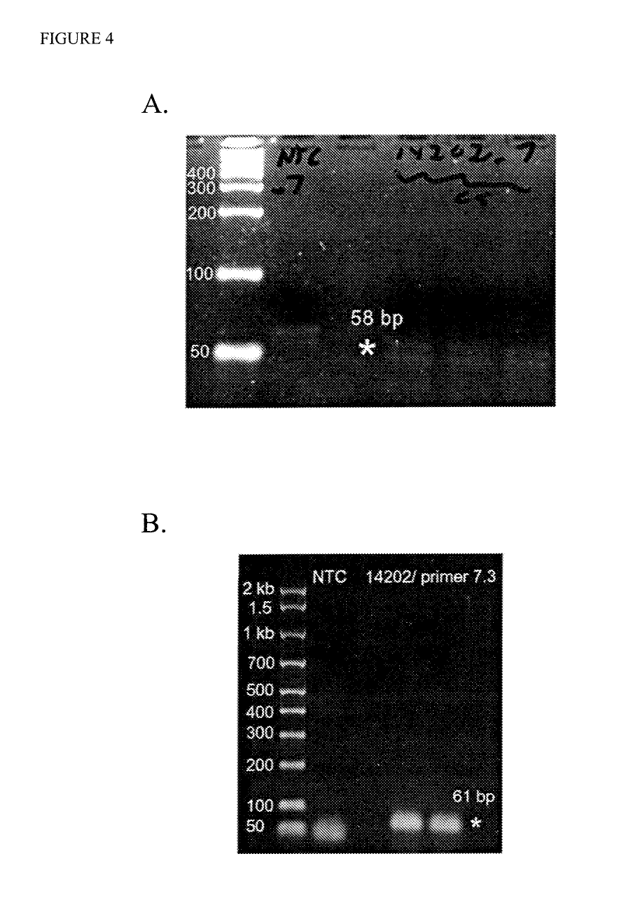

FIG. 4A shows an electrophoretic gel of sample 14202 amplified with RhD primer set 7 (last 3 lanes) (Ct 37.3). Two closely sized amplicons of approximately 53 and 58 bp are visible on the 4.5% MS8 agarose gel. Both of these products were cloned separately into the TA cloning vector and multiple clones were sequenced. All sequenced clones revealed the same, correct amplicon of 58 bp, which was identified using BLAST as the RhD locus.

Sequencing: Homo sapiens Rh Blood Group, D Antigen (RHD), mRNA

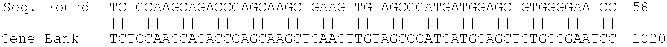

### Rh(D) Primer Set 7.3 (Exon 7)

FIG. 4B depicts the electrophoretic analysis of sample 14202 amplified with RhD primer set 7.3 (last two lanes) (Ct 38.2). There is one amplicon of approximately 61 bp which was identified using BLAST as the RhD locus:

Sequencing: Homo sapiens Rh Blood Group, D Antigen (RHD), mRNA

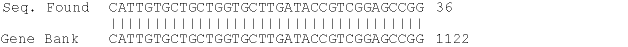

### Rh(D) Primer Sets 10 and 10.1

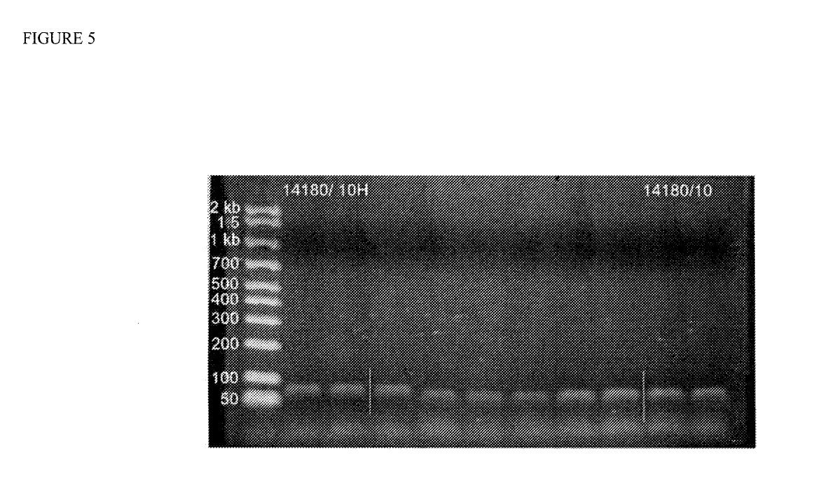

FIG. 5 shows sample 14180 amplified with RhD primer sets 10 (Ct 42.1, last 2 lanes, 59 bp) and 10.1 (10H; Ct 37.7, first two lanes, 74 bp). The amplicons of approximately 74 bp and 59 bp for primer set 10.1 and 10, respectively were identified using BLAST as the RhD locus:

### RhD Primer Set 10 (Exon 10):

Sequencing: Homo sapiens Rh Blood Group, D Antigen (RHD), mRNA

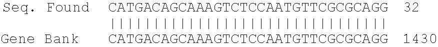

### RhD Primer Set 10H (Exon 10):

Sequencing: Homo sapiens Rh Blood Group, D Antigen (RHD), mRNA

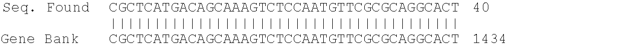

The results of these experiments, summarized in Table 3, confirm that all high Ct PCR products, up to a Ct value of 42.1, resulting from real-time PCR performed with RhD primers, are real amplified products.

TABLE 3
Ct validation results for RhD primer sets

| RhD | Amplicon | | | | |
|---|---|---|---|---|---|
| Exon | size | Sample | Ct | Sequence identified | Method |
| 4.2 | 70 | 14202 | 40.2 | RhD locus- 100% match | cloning |
| 4.3 | 62 | 14202 | 40.4 | RhD locus- 100% match | PCR |
| 5 | 83 | 14180 | 39.3 | RhD locus- 100% match | PCR |
| 5.2 | 73 | 14180 | 35 | RhD locus- 100% match | PCR |
| 7 | 58 | 14202 | 37.3 | RhD locus- 100% match | cloning |
| 7.3 | 61 | 14202 | 38.2 | RhD locus- 100% match | PCR |
| 10 | 59 | 14180 | 42.1 | RhD locus- 100% match | PCR |
| 10.1 | 74 | 14180 | 37.7 | RhD locus- 100% match | PCR |

### Example 3

### Determination of the Presence of Fetal DNA in Maternal Blood Samples

### Fetal RhD Negative Male Samples

The objective of the experiments described in this example was to confirm the presence of fetal DNA in lysates prepared from maternal blood samples. Fetal RhD positivity from RhD negative mothers' blood was considered diagnostic of the presence of fetal DNA in the maternal samples. In cases where the fetal RhD status was negative, fetal origin of DNA was established first by determining the fetal gender by RT-PCR with primers and probes designed to amplify SRY (sex-determining region) and FCY loci on the Y-chromosome. The FCY primers and probe that were used were previously described in the literature (D. Bianchi, et al., (2001) Clin. Chem., Vol. 47: 1867). Fetal gender was also determined using novel primers and probe developed to amplify the DAZ (deleted in azoospermia) gene on the Y-chromosome. Beta-globin gene was used as a house keeping gene along with known male DNA as a positive control and female DNA as a negative control. Ct values for SRY positive samples ranged from 32 to 37.5 while FCY positive samples gave Ct values between 32 to 38. Ct values for DAZ positive samples were in the range of 30 to 35 indicating that the DAZ primers and probe were more sensitive than the SRY and FCY primers/probes. Beta-globin values ranged from 24 to 32. The sequences of SRY, FCY, DAZ and beta-globin gene primers and probes are listed below:

5′-------------------------------------------------3′
SRY Forward Primer
(SEQ ID NO: 25)
TGCACAGAGAGAAATACCCGAATTA
SRY Reverse Primer
(SEQ ID NO: 26)
TGCAATTCTTCGGCAGCAT
SRY Probe
(SEQ ID NO: 27)
(6FAM)-AAGTATCGACCTCGTCGGAAGGCGAA-(MGBNFQ)
FCY Forward Primer
(SEQ ID NO: 28)
TCCTGCTTATCCAAATTCACCAT
FCY Reverse Primer
(SEQ ID NO: 29)
ACTTCCCTCTGACATTACCTGATAATTG
FCY Probe
(SEQ ID NO: 30)
(6FAM)-AAGTCGCCACTGGATATCAGTTCCCTTGT-(TAMRA)
DAZ 1.3 Forward primer
(SEQ ID NO: 31)
CGTATTCATTTTTTTCTGGAACCTTT
DAZ 1.3 reverse Primer
(SEQ ID NO: 32)
CTGATATCCAGTGGCGACTTGA
DAZ Probe
(SEQ ID NO: 33)
(6FAM)-CAGGCATTTCCTGCTTATCCAAATTCACC-(BHQ-1)
Beta-Globin Forward Primer
(SEQ ID NO: 34)
GTGCACCTGACTCCTGAGGAGA
Beta-Globin Reverse Primer
(SEQ ID NO: 35)
CCTTGATACCAACCTGCCCAG
Beta-Globin Probe
(SEQ ID NO: 36)
(6FAM)-AAGGTGAACGTGGATGAAGTTGGTGG-(TAMRA)

### Fetal RhD Negative Female Samples

Samples that were RhD negative and also negative for the Y-chromosome genes were analyzed by RT-PCR using 16 sets of polymorphic markers designed to amplify paternally inherited alleles by the fetus (M. Alizadeh, et al., (2002), Blood, Vol. 99: 4618). First, these bi-allelic markers were tested on maternal DNA. Then, the fetal DNA was tested for those markers that were negative on maternal DNA. The presence of an allele in fetal DNA and absence of the same allele in maternal genome was indicative of the paternally inherited allele by the fetus. Sequences of the primers and probes for the detection of these markers are shown below:

5′------------------------------------------3′
S01-Forward Primer
(SEQ ID NO: 37)
GGTACCGGGTCTCCACATGA
S01-Reverse Primer
(SEQ ID NO: 38)
GGGAAAGTCACTCACCCAAGG
S01-Probe
(SEQ ID NO: 39)
(6FAM)-CTGGGCCAGAATCTTGGTCCTCACA-(BHQ)
S02-Forward Primer
(SEQ ID NO: 40)
GCTTCTCTGGTTGGAGTCACG
S02-Reverse Primer
(SEQ ID NO: 41)
GCTTGCTGGCGGACCCT
S02-Probe
(SEQ ID NO: 42)
(6FAM)-CTGCACCACCAAATCATCCCCGTG-(BHQ)
5′-----------------------------------------------3′
S03-Forward Primer
(SEQ ID NO: 43)
CTTTTGCTTTCTGTTTCTTAAGGGC
S03-Reverse Primer
(SEQ ID NO: 44)
TCAATCTTTGGGCAGGTTGAA
S03-probe
(SEQ ID NO: 45)
(6FAM)-CATACGTGCACAGGGTCCCCGAGT-(BHQ)
S04-Forward Primer
(SEQ ID NO: 46)
CTGGTGCCCACAGTTACGCT
S04-Reverse Primer
(SEQ ID NO: 47)
AAGGATGCGTGACTGCTATGG
S04-Probe
(SEQ ID NO: 48)
(6FAM)-TCCTGGCAGTGTGGTCCCTTCAGAA-(BHQ)
S05-Forward Primer
(SEQ ID NO: 49)
AAAGTAGACACGGCCAGACTTAGG
S05-Reverse Primer
(SEQ ID NO: 50)
CATCCCCACATACGGAAAAGA
S05-Probe
(SEQ ID NO: 51)
(6FAM)-CCCTGGACACTGAAAACAGGCAATCCT-(BHQ)
S06-Forward Primer
(SEQ ID NO: 52)
CAGTCACCCCGTGAAGTCCT
S06-Reverse Primer
(SEQ ID NO: 53)
TTTCCCCCATCTGCCTATTG
S06-Probe
(SEQ ID NO: 54)
(6FAM)-CCCATCCATCTTCCCTACCAGACCAGG-(BHQ)
S07-Forward Primer
(SEQ ID NO: 55)
TGGTATTGGCTTTAAAATACTGGG
S07-Reverse Primer
(SEQ ID NO: 56)
TGTACCCAAAACTCAGCTGCA
S07-Probe
(SEQ ID NO: 57)
(6FAM)-TCCTCACTTCTCCACCCCTAGTTAAACAG-(BHQ)
S07b-Forward Primer
(SEQ ID NO: 58)
GGTATTGGCTTTAAAATACTCAACC
S07b-Reverse Primer
(SEQ ID NO: 59)
CAGCTGCAACAGTTATCAACGTT
S07-Probe
(SEQ ID NO: 57)
(6FAM)-TCCTCACTTCTCCACCCCTAGTTAAACAG-(BHQ)
S08-Forward Primer
(SEQ ID NO: 60)
CTGGATGCCTCACTGATCCA
S08-Reverse Primer
(SEQ ID NO: 61)
TGGGAAGGATGCATATGATCTG
S08-probe
(SEQ ID NO: 62)
(6FAM)-CTCCCAACCCCCATTTCTGCCTG-(BHQ)
S08b-Forward Primer
(SEQ ID NO: 63)
GCTGGATGCCTCACTGATGTT
S08-Reverse Primer
(SEQ ID NO: 61)
TGGGAAGGATGCATATGATCTG
S08-Probe
(SEQ ID NO: 62)
(6FAM)-CTCCCAACCCCCATTTCTGCCTG-(BHQ)
S09a-Forward Primer
(SEQ ID NO: 64)
GGGCACCCGTGTGAGTTTT
S09a-Reverse Primer
(SEQ ID NO: 65)
TCAGCTTGTCTGCTTTCTGGAA
S09-Probe
(SEQ ID NO: 66)
(6FAM)-TGGAGGATTTCTCCCCTGCTTCAGACAG-(BHQ)
S09a-Forward Primer
(SEQ ID NO: 64)
GGGCACCCGTGTGAGTTTT
S09b-Reverse Primers
(SEQ ID NO: 67)
CAGCTTGTCTGCTTTCTGCTG
S09-Probe
(SEQ ID NO: 66)
(6FAM)-TGGAGGATTTCTCCCCTGCTTCAGACAG-(BHQ)
5′-----------------------------------------------3′
S10a-Forward Primer
(SEQ ID NO: 68)
GCCACAAGAGACTCAG
S10a-Reverse Primer
(SEQ ID NO: 69)
TGGCTTCCTTGAGGTGGAAT
S10-Probe
(SEQ ID NO: 70)
(6FAM)-CAGTGTCCCACTCAAGTACTCCTTTGGA-(BHQ)
S10b-Forward Primer
(SEQ ID NO: 71)
TTAGAGCCACAAGAGACAACCAG
S10a-Reverse Primer
(SEQ ID NO: 69)
TGGCTTCCTTGAGGTGGAAT
S10-Probe
(SEQ ID NO: 70)
(6FAM)-CAGTGTCCCACTCAAGTACTCCTTTGGA-(BHQ)
S11a-Forward Primer
(SEQ ID NO: 72)
TAGGATTCAACCCTGGAAGC
S11a-Reverse Primer
(SEQ ID NO: 73)
CCAGCATGCACCTGACTAACA
S11-Probe
(SEQ ID NO: 74)
(6FAM)-CAAGGCTTCCTCAATTCTCCACCCTTCC-(BHQ)
S11b-Forward Primer
(SEQ ID NO: 75)
CCCTGGATCGCCGTGAA
S11a-Reverse Primer
(SEQ ID NO: 73)
CCAGCATGCACCTGACTAACA
S11-Probe
(SEQ ID NO: 74)
(6FAM)-CAAGGCTTCCTCAATTCTCCACCCTTCC-(BHQ)

Sequencing of the amplicons with Ct value as high as 43 has shown beyond the shadow of a doubt that these high Ct amplicons are indeed real PCR products (data not shown). The results of these experiments demonstrate that fetal DNA can be extracted by the selective lysis of maternal blood samples as described in Example 1 and that this isolated DNA can be used to accurately predict the RhD genotype of the fetus.

### Example 4

### Novel Primers and Probes for Amplifying Particular Exons of the RhD Gene

This example describes additional novel primer and probe sequences for amplifying and detecting particular exons of the human RhD gene. These probe and primers sequences are used in methods of determining a subject's RhD genotype, particularly in RT-PCR-based methods.

5′------------------------------------------3′
RhD Exon 2.2 Forward Primer
(SEQ ID NO: 76)
CCGTGATGGCGGCCA
RED Exon 2.2 Reverse Primer
(SEQ ID NO: 77)
CAGCTGTGTCTCCGGAAACTC
RhD Exon 2.2 Probe
(SEQ ID NO: 78)
(NED)-CTTGGGCTTCCTCACCT-(MGBNFQ)
5′-----------------------------------------------3′
RhD Intron 4 Forward Primer
(SEQ ID NO: 79)
ACAAGGAAACAAAGGCCAAGAG
RhD Intron 4 Reverse Primer
(SEQ ID NO: 80)
AATTAAGCACTTCACAGAGCAGGTT
RhD Intron 4 Probe
(SEQ ID NO: 81)
(6FAM)-TTGAAATCTGCATACCCCAGGCCTCCT-(MGBNFQ)
RhD Intron 4 Forward Primer
(SEQ ID NO: 79)
ACAAGGAAACAAAGGCCAAGAG
RhD Intron 4 Reverse Primer
(SEQ ID NO: 80)
AATTAAGCACTTCACAGAGCAGGTT
RhD Intron 4 Probe
(SEQ ID NO: 82)
(6FAM)-TTGAAATCTGCATACCCCAGGCCTCCT-(BHQ)
RhCED Intron 4.1 Forward Primer
(SEQ ID NO: 83)
AGGCTGAGGCAGGAGAATCTT
RhCED Intron 4.1 Reverse Primer
(SEQ ID NO: 84)
GCAGTGGCGCGATCTTG
RhCED Intron 4.1 Probe
(SEQ ID NO: 85)
(6FAM)-TGAATCCAGGTGGTGGAGGTTGCA-(MGBNFQ)
RhD Intron 4.1 Forward Primer
(SEQ ID NO: 86)
TGAGTAGTGTTTGCTAAATTCATACCTTT
RhD Intron 4.1 Reverse primer
(SEQ ID NO: 87)
ACCCCAGGCCTCCTGAAC
RhD Intron 4.1 Probe
(SEQ ID NO: 88)
(6FAM)-TAAGCACTTCACAGAGCAG-(BHQ)
RhDΨ Exon 4.2 Forward Primer
(SEQ ID NO: 89)
GCATGGCAGACAAACTGGGTAAT
RhDΨ Exon 4.2 Reverse Primer
(SEQ ID NO: 90)
CTGCCAAAGCCTCTACCGG
RhDΨ Exon 4.2 Probe
(SEQ ID NO: 91)
(6FAM)-TTGCTGTCTGATCTTT-(BHQ)
RHDΨ Exon 5.1 Forward Primer
(SEQ ID NO: 92)
ATGTTCTGGCCAAGTTTCAAGAT
RHDΨ Exon 5.1 Reverse Primer
(SEQ ID NO: 93)
GCTACAGCATAGTAGGTGTTGAAGTC
RHDΨ Exon 5.1 Probe
(SEQ ID NO: 94)
(6FAM)-CTCTGCTGAGAAGTCCAATCGAAAGGAAGA-(BHQ)
RHDΨ Exon 5.1 Forward Primer
(SEQ ID NO: 95)
ATGTTCTGGCCAAGTTTCAACGT
RHDΨ Exon 5.1 Reverse Primer
(SEQ ID NO: 96)
GCTACAGCATAGTAGGTGTTGAACGC
RHDΨ Exon 5.1 Probe
(SEQ ID NO: 97)
(6FAM)-CTCTGCTGAGAAGTCCAATCGAAAGGAAGA-(MGBNFQ)
RHDψ Exon 5.1 Forward Primer
(SEQ ID NO: 98)
GATGTTCTGGCCAAGTTTCAACTC
RHDΨ Exon 5.1 Reverse Primer
(SEQ ID NO: 99)
CTGCTACAGCATAGTAGGTGTTGAACAC
RHD ψ Exon 5.1 Probe
(SEQ ID NO: 94)
(6FAM)-CTCTGCTGAGAAGTCCAATCGAAAGGAAGA-(BHQ)
5′-----------------------------------------------3′
RHDΨ Exon 5.1 Forward Primer
(SEQ ID NO: 100)
ATGTTCTGGCCAAGTTTCAACAT
RHDΨ Exon 5 Reverse Primer
(SEQ ID NO: 101)
GCTACAGCATAGTAGGTGTTGAACTC
RHDΨ Exon 5.1 Probe
(SEQ ID NO: 94)
(6FAM)-CTCTGCTGAGAAGTCCAATCGAAAGGAAGA-(BHQ)
RhD Exon 5.2 Forward Primer
(SEQ ID NO: 102)
AATAAATCATAATGTGGATGTTCTGGCCAAGTT
RhD Exon 5.2 Reverse Primer
(SEQ ID NO: 103)
AATAAATCATAATGAACACGGCATTCTTCCTTTC
RhD Exon 5.2 Probe
(SEQ ID NO: 12)
(6FAM)-AACTCTGCTCTGCTGAGAAGTCCAAT-(BHQ)
RHDΨ Exon 5.2 Forward Primer
(SEQ ID NO: 104)
GCGCCCTCTTCTTGTGGAAC
RHDΨ Exon 5.2 Reverse Primer
(SEQ ID NO: 105)
CATTCTTCCTTTCGATTGGACTTCT
RHDΨ Exon 5.2 Probe
(SEQ ID NO: 106)
(6FAM)-TCTGGCCAAGTTTC-(BHQ)
RHDΨ Exon 5.3 Forward Primer
(SEQ ID NO: 10)
TGTGGATGTTCTGGCCAAGTT
RHDΨ Exon 5.3 Reverse Primer
(SEQ ID NO: 107)
TTGAACACGGCATTCTTCCTT
RHDΨ Exon 5.3 Probe
(SEQ ID NO: 108)
(6FAM)-CAACTCTGCTCTGCTGAGAAGTCCAATCG-(BHQ)
RHDΨ Exon 6.1 Forward Primer
(SEQ ID NO: 109)
CACACGCTATTTCTTTGCAGACTTAT
RHDΨ Exon 6.1 Reverse Primer
(SEQ ID NO: 110)
GTTGTCTAGTTTCTTACCGGCAGGT
RHDΨ Exon 6.1 Probe
(SEQ ID NO: 111)
(6FAM)-TGTCACCTGATCCCTTCTCCGTGGC-(BHQ)
RHDΨ Exon 6.1 Forward Primer
(SEQ ID NO: 112)
ACGCTATTTCTTTGCAGACTTGG
RHDΨ Exon 6.1 Reverse Primer
(SEQ ID NO: 113)
GTTGTCTAGTTTCTTACCGGCAGTT
RHDΨ Exon 6.1 Probe
(SEQ ID NO: 111)
(6FAM)-TGTCACCTGATCCCTTCTCCGTGGC-(BHQ)
RHDΨ Exon 6.1 Forward Primer
(SEQ ID NO: 114)
ACGCTATTTCTTTGCAGACTATG
RHDΨ Exon 6.1 Reverse Primer
(SEQ ID NO: 115)
GTTGTCTAGTTTCTTACCGGCACCT
RHDΨ Exon 6.1 Probe
(SEQ ID NO: 111)
(6FAM)-TGTCACCTGATCCCTTCTCCGTGGC-(BHQ)
RHDΨ Exon 6.1 Forward Primer
(SEQ ID NO: 116)
ACGCTATTTCTTTGCAGACTTTG
RHDΨ Exon 6.1 Reverse Primer
(SEQ ID NO: 117)
GTTGTCTAGTTTCTTACCGGCAGCT
RHDΨ Exon 6.1 Probe
(SEQ ID NO: 111)
(6FAM)-TGTCACCTGATCCCTTCTCCGTGGC-(BHQ)
5′------------------------------------------3′
RhD Exon 7.2 Forward Primer
(SEQ ID NO: 118)
CAGCTCCATCATGGGCTACAA
RhD Exon 7.2 Reverse Primer
(SEQ ID NO: 119)
GCACCAGCAGCACAATGTAGA
RhD Exon 7.2 Probe
(SEQ ID NO: 120)
(6FAM)-CTTGCTGGGTCTGCTTGGAGAG-(BHQ)
RhD Exon 10.3 Forward Primer
(SEQ ID NO: 121)
GCAGTGCCGCAATCTCG
RhD Exon 10.3 Reverse Primer
(SEQ ID NO: 122)
CTGAGGCAGGAGAATTGCTTG
RhD Exon 10.3 Probe
(SEQ ID NO: 123)
(6FAM)-AACCTCCGCCTCCCA-(MGBNFQ)

It is understood that the disclosed invention is not limited to the particular methodology, protocols and materials described as these may vary. It is also understood that the terminology used herein is for the purposes of describing particular embodiments only and is not intended to limit the scope of the present invention which will be limited only by the appended claims.

Those skilled in the art will recognize, or be able to ascertain using no more than routine experimentation, many equivalents to the specific embodiments of the invention described herein. Such equivalents are intended to be encompassed by the following claims.

1. An isolated polynucleotide comprising a sequence selected from the group consisting of SEQ ID NO: 1, SEQ ID NO: 2, SEQ ID NO: 4, SEQ ID NO: 5, SEQ ID NO: 10, SEQ ID NO: 11, SEQ ID NO: 13, SEQ ID NO: 14, SEQ ID NO: 17, SEQ ID NO: 19, SEQ ID NO: 20, SEQ ID NO: 22, and SEQ ID NO: 23, wherein the isolated polynucleotide contains less than fifty bases.

2. (canceled)

3. An isolated polynucleotide comprising a sequence selected from the group consisting of SEQ ID NO: 3, SEQ ID NO: 6, SEQ ID NO: 9, SEQ ID NO: 12, SEQ ID NO: 15, SEQ ID NO: 18, SEQ ID NO: 21, and SEQ ID NO: 24, wherein the isolated polynucleotide contains less than fifty bases.

4. The isolated polynucleotide of claim 3, wherein a reporter molecule is attached to the 5′ end of the polynucleotide and a quencher molecule is attached to the 3′ end of the polynucleotide.

5. (canceled)

6. An isolated polynucleotide comprising a sequence recited in SEQ ID NO: 31, SEQ ID NO: 32, or SEQ ID NO: 33, wherein the isolated polynucleotide contains less than fifty bases.

7. (canceled)

8. A RhD genotyping kit comprising:
at least one primer set, wherein said at least one primer set comprises a forward primer and a reverse primer;
at least one labeled probe; and
instructions for using said at least one primer set and said at least one probe for detecting a RHD gene in a biological sample, wherein said forward primer and said reverse primer hybridize to an exon of the human RHD gene.

9-21. (canceled)

22. The kit of claim 8, wherein said kit comprises two or more primer sets and two or more labeled probes.

23-24. (canceled)

25. The kit of claim 8, further comprising a lysis reagent.

26-27. (canceled)

28. A method of determining RhD genotype of a subject comprising:
lysing cells in a biological sample to form a lysing mixture, wherein said biological sample contains one or more cells from the subject;
extracting nucleic acid from said lysing mixture; and
detecting at least one exon of the RHD gene in said extracted nucleic acid, wherein the presence or absence of said exon indicates the subject's RhD genotype.

29-35. (canceled)

36. The method of claim 28, wherein said detection of at least one exon of the RHD gene comprises amplifying said at least one exon with one or more primer sets and identifying the at least one exon with one or more labeled probes.

37-50. (canceled)

51. The method of claim 36, wherein two or more primer sets are used to amplify said at least one exon and two or more labeled probes are used to identify said at least one exon.

52-61. (canceled)

62. A method of determining RhD genotype of a subject comprising:
extracting nucleic acid from a biological sample, wherein said biological sample contains one or more cells from the subject; and
detecting at least three exons of the RHD gene in said extracted nucleic acid, wherein the presence or absence of said exons indicates the subject's RhD genotype.

63. The method of claim 62, wherein said detection of at least three exons of the RHD gene comprises amplifying said at least three exons with three or more primer sets and identifying the at least three exons with three or more labeled probes.

64-73. (canceled)

74. A reagent mixture comprising isolated nucleic acid; three or more primer sets for amplification of three or more exons of a RHD gene, wherein each said primer set comprises a forward primer and a reverse primer; and three or more labeled probes.

75. The reagent mixture of claim 74, wherein said three or more exons are selected from the group consisting of exon 4, exon 5, exon 7, and exon 10 of the human RHD gene.

76. The reagent mixture of claim 75, wherein the forward primers of said three or more primer sets are selected from the group consisting of SEQ ID NO: 1, SEQ ID NO: 4, SEQ ID NO: 7, SEQ ID NO: 10, SEQ ID NO: 13, SEQ ID NO: 16, SEQ ID NO: 19, and SEQ ID NO: 22.

77. The reagent mixture of claim 75, wherein said reverse primers of said three or more primer sets are selected from the group consisting of SEQ ID NO: 2, SEQ ID NO: 5, SEQ ID NO: 8, SEQ ID NO: 11, SEQ ID NO: 14, SEQ ID NO: 17, SEQ ID NO: 20, and SEQ ID NO: 23.

78. The reagent mixture of claim 75, wherein said three or more labeled probes are selected from the group consisting of SEQ ID NO: 3, SEQ ID NO: 6, SEQ ID NO: 9, SEQ ID NO: 12, SEQ ID NO: 15, SEQ ID NO: 18, SEQ ID NO: 21, and SEQ ID NO: 24.

79-80. (canceled)
